SEQUENCE LISTING (12 sequences)

SEQ ID NO: 1 - DNA, 24 bp, Artificial Sequence
  synthetic primer
tggtctacgtagcctcacttcact

SEQ ID NO: 2 - DNA, 20 bp, Artificial Sequence
  synthetic primer
tggaggcgacatcaattcct

SEQ ID NO: 3 - DNA, 20 bp, Artificial Sequence
  Synthetic Primer
cagagccgagaggactcgaa

SEQ ID NO: 4 - DNA, 18 bp, Artificial Sequence
  Synthetic Primer
ccggttgacgcggagtag

SEQ ID NO: 5 - DNA, 20 bp, Artificial Sequence
  Synthetic Primer
agcatgcactccttacagca

SEQ ID NO: 6 - DNA, 20 bp, Artificial Sequence
  Synthetic Primer
acaaagcactctgtgccagc

SEQ ID NO: 7 - DNA, 19 bp, Artificial Sequence
  Synthetic Primer
acagggtggcgatgtacga

SEQ ID NO: 8 - DNA, 21 bp, Artificial Sequence
  Synthetic Primer
gcacccatagaccgaggtgat

SEQ ID NO: 9 - DNA, 18 bp, Artificial Sequence
  Synthetic Primer
gtgcctgctcagtgccag

SEQ ID NO: 10 - DNA, 18 bp, Artificial Sequence
  Synthetic Primer
cgctcgtccggaaaagtt

SEQ ID NO: 11 - DNA, 19 bp, Artificial Sequence
  Synthetic Primer
cagctggatgcccatgttc

SEQ ID NO: 12 - DNA, 18 bp, Artificial Sequence
  Synthetic Primer
agccgcagcagaaaatgc

(19) United States
(12) Patent Application Publication
(10) Pub. No.: US 2015/0164991 A1
(43) Pub. Date: Jun. 18, 2015

(54) Method and Kit for Evaluating and Monitoring a Treatment Program for Anemia

(71) Applicant: Vanderbilt University, Nashville, TN (US)

(72) Inventors: Volker H. Haase, Nashville, TN (US); Qingdu Liu, Nashville, TN (US)

(21) Appl. No.: 14/531,999

(22) Filed: Nov. 3, 2014

Related U.S. Application Data
(60) Provisional application No. 61/898,697, filed on Nov. 1, 2013.

(51) Int. Cl. A61K 38/18; A61K 33/26

(52) U.S. Cl. CPC A61K 38/1816; A61K 33/26

(57) ABSTRACT

The present disclosure relates to methods and kits for evaluating and/or monitoring a treatment program, such as in an anemic subject or in a subject receiving treatment for anemia, including determining a presence or an amount of growth differentiation factor 15 (GDF15) and/or hepcidin in a biological sample from the subject. Further, in some embodiments, the disclosure provides methods and/or kits for treating a disorder of iron homeostasis in a subject, comprising measuring an amount of GDF15 and/or an amount of hepcidin in a sample from a subject and administering iron and/or erythropoietin (EPO) to the subject if there is a measurable difference in the amount of GDF15 and/or hepcidin in the sample as compared to a reference amount.

### RELATED APPLICATION

This application claims priority from U.S. Provisional Application Ser. No. 61/898,697, filed Nov. 1, 2013, the entire disclosure of which is incorporated herein by this reference.

### GOVERNMENT INTEREST

The invention(s) of the present disclosure was made with government support under R01-DK080821 awarded by the National Institutes of Health. The government has certain rights in the invention(s).

### TECHNICAL FIELD

The presently-disclosed subject matter relates to tools useful for evaluating and/or monitoring a treatment program, such as a program for treating a disorder of iron homeostasis in a subject. In particular, the presently-disclosed subject matter relates to methods and kits for evaluating and/or monitoring a treatment program, such as in an anemic subject or in a subject receiving treatment for anemia, including determining a presence or an amount of growth differentiation factor 15 and/or hepcidin in a biological sample from the subject.

### BACKGROUND

Iron demand in bone marrow increases when erythropoiesis is stimulated by hypoxia via increased erythropoietin (EPO) synthesis in kidney and liver. Hepcidin, a small polypeptide produced by hepatocytes, plays a central role in regulating iron uptake and utilization. It promotes internalization and degradation of ferroportin, the only known cellular iron exporter. Hypoxia suppresses hepcidin, thereby enhancing intestinal iron uptake and release from internal stores. While hypoxia-inducible factor (HIF), a central mediator of cellular adaptation to hypoxia, directly regulates renal and hepatic EPO synthesis under hypoxia, the molecular basis of hypoxia/HIF-mediated hepcidin suppression in the liver remains unclear.

### BRIEF SUMMARY

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of features.

The present disclosure provides, in certain embodiments, a method for treating a disorder of iron homeostasis in a subject. The method may comprise, for example, the steps of (i) providing a biological sample from the subject; (ii) determining an amount of growth differentiation factor 15 (GDF15) in the sample; (iii) comparing the amount of the GDF15 to a reference amount of GDF15; and/or (iv) administering to the subject an effective amount of at least one of iron and erythropoietin (EPO) if there is a measurable difference in the amount of GDF15 in the sample as compared to the reference amount of GDF15. In some embodiments, the subject is a human, and in certain embodiments, the sample comprises a blood sample and/or a serum sample. Further, in some embodiments, the effective amount of iron and/or EPO may be administered to the subject on an hourly, daily and/or weekly basis. In certain embodiments, the disorder of iron homeostasis comprises anemia. And in some embodiments, the method may include a step of monitoring a bone marrow and/or an erythroid response in the subject and/or a step of determining a red blood cell count in a sample obtained from the subject.

Moreover, in certain embodiments, the subject may be a human subject. And in some embodiments, the subject may be receiving dialysis, may be resistant to treatment with recombinant EPO and/or is not responding to treatment with recombinant EPO, and/or may be receiving treatment with recombinant erythropoietin, a hypoxia-inducible factor (HIF)-stabilizing composition, and/or a composition that stimulates endogenous EPO synthesis. In still further embodiments, the method of the present disclosure may further comprise the steps of: (i) determining an amount of hepcidin in a sample; (ii) comparing the amount of hepcidin in the sample to a reference amount of hepcidin; and/or (iii) determining a ratio of the amount of GDF15 in the sample to the amount of hepcidin in the sample.

The present disclosure additionally provides, in certain embodiments, a method for treating disorder of iron homeostasis in a subject, the method comprising: (i) providing a biological sample from the subject; (ii) determining an amount of hepcidin in the sample; (iii) comparing the amount of the hepcidin to a reference amount of hepcidin; and/or (iv) administering to the subject an effective amount of at least one of iron and erythropoietin (EPO) if there is a measurable difference in the amount of hepcidin in the sample as compared to the reference amount of hepcidin. In some embodiments, the effective amount of at least one of iron and EPO is administered to the subject on an hourly, daily and/or weekly basis. And in a particular embodiment, the disorder of iron homeostasis comprises anemia.

Meanwhile, in some embodiments, the present disclosure provides a method for evaluating and/or monitoring a treatment program for a subject, the method comprising: (i) providing a biological sample from the subject; (ii) determining a presence or an amount of GDF15 in the sample and/or determining a presence or an amount of hepcidin in the sample; and/or (iii) comparing the presence or the amount of the GDF15 and/or hepcidin to a reference, wherein the treatment program is evaluated based on a measurable difference in the presence or the amount of the GDF15 and/or hepcidin as compared to the reference. In certain embodiments, the subject is: (i) receiving treatment with recombinant EPO, a HIF-stabilizing composition, and/or a composition that stimulates endogenous EPO synthesis; (ii) anemic and/or receiving treatment for anemia; (iii) receiving dialysis; (iv) receiving renal dialysis; (v) resistant to treatment with recombinant EPO; and/or (vi) not responding to treatment with recombinant EPO. Additionally, in some embodiments, the methods of the present disclosure comprise a step of: (i) determining a ratio of an amount of GDF15 to an amount of hepcidin in a sample; (ii) monitoring at least one bone marrow and/or erythroid response in a subject; and/or (iii) predicting a bone marrow response to a treatment program, such as a pharmacologic treatment program designed to improve iron utilization by bone marrow or correction of functional iron-deficiency anemia.

And in some embodiments, the reference comprises (i) a control and/or (ii) a level of the GDF15 and/or hepcidin in a sample from a subject taken over a time course and/or at pre-determined intervals of time. In further embodiments, the reference comprises a sample from the subject collected prior to initiation of a treatment program. And in some embodiments, the biological sample is collected after initiation of the treatment program. In certain embodiments, the reference comprises a standard sample. And in some embodiments, the reference comprises control data. In still further embodiments, the reference comprises a level of the GDF15 and/or hepcidin in one or more samples from one or more individuals who are known responders or who are known non-responders to treatment with recombinant erythropoietin, a HIF-stabilizing composition and/or a composition that stimulates endogenous EPO synthesis.

In certain embodiments, the methods of the present disclosure include a step of alternating a treatment and/or alternating a treatment program. In some embodiments, alternating a treatment program involves administering iron and/or EPO to a subject and/or altering the dose of iron and/or EPO being administered to the subject. Certain embodiments of the methods of the present disclosure may be performed and/or carried out in vitro.

And in some embodiments, the present disclosure provides a kit for evaluating and/or monitoring a treatment program for a disorder of iron homeostasis.

### BRIEF DESCRIPTION OF THE DRAWINGS

FIG. 1 shows the inactivation of Vhl suppresses hepcidin. Shown are results from real-time PCR analysis of Vhl and Vegf mRNA levels in Vhl^{-/-} livers (n=3) and Epo mRNA levels in Vhl−/− kidneys and livers (n=3); analysis was performed on day 8 after the first tamoxifen injection. Relative mRNA expression levels were normalized to 18S ribosomal RNA.

FIG. 2 illustrates the global inactivation of Vhl induces erythropoiesis. Shown are individual hematocrit values (n=14 and 13 respectively), reticulocyte counts (%) (n=6 each), serum Epo concentrations (sEpo) (n=3 each) from control and mutant mice and a representative picture of a control and a Vhl^{-/-} spleen. Lower right panel shows a representative FACS plot of CD71/Ter119 double stained bone marrow (BM) and spleen cells from an individual control mouse and Vhl mutant. Percentages of CD71^{high}/Ter119^{high}-positive cells (right upper quadrant) are indicated.

FIG. 3 provides the relative expression levels of hepcidin mRNA in control and Vhl^{-/-} livers (n=5 and 3 respectively), serum iron (n=3 each) and liver iron concentrations (n=7 and 4 respectively). H-ferritin protein levels in control and Vhl^{-/-} livers were determined by immunoblot in 3 mice, β-actin served as loading control. Asterisks indicate a statistically significant difference when comparisons were made to the control group, *P<0.05; **P<0.01 and ***P<0.001. Shown are arithmetic mean values±SEM. Abb.: Co, Cre-negative littermate control; Hct, hematocrit; retic, reticulocytes.

FIG. 4 illustrates hepcidin suppression in Vhl^{-/-} livers is Hif-dependent. Indeed, FIG. 4 provides teal-time PCR analysis of Vhl, Vegf, Epo, hepcidin, Dmt1 and Trfc expression in Vhl/Hif-1a/Hif-2a^{-/-} livers. Relative mRNA expression levels were normalized to 18S ribosomal RNA. Bars represent arithmetic mean values±SEM (n=3).

FIG. 5 shows serum Epo concentrations and serum iron levels in Vhl/Hif-1a/Hif-2a^{-/-} mice (n=3). Circles and squares represent data points for individual mice. Error bars represent SEM, *, P<0.05; **, P<0.01 for comparisons to controls. Abb.: Co, Cre-negative littermate control.

FIG. 6 illustrates that hepatocyte-specific inactivation of Phd2 does not suppress hepcidin. Hif-1α and Hif-2α protein levels in Phd2^{-/-} livers are shown. Ponceau staining is used to assess for equal protein loading. +Co indicates a positive control sample obtained from Vhl−/− livers.

FIG. 7 shows that hepatocyte-specific inactivation of Phd2 does not increase Epo mRNA and does not suppress hepcidin mRNA levels in Phd2^{-/-} livers. Shown are relative mRNA expression levels normalized to 18S ribosomal RNA in mutant and control livers. Corresponding renal Epo mRNA levels are shown for comparison (n=3).

FIG. 8 presents hematocrit, reticulocyte counts, serum Epo (sEpo) and serum iron levels in control and Phd2 mutant mice (n=3 each). Shown are mean values±SEM. For statistical analysis mutants were compared to controls. Abb.: Co, Cre-negative littermate control; Hct, hematocrit; retic, reticulocytes.

FIG. 9 shows that Hif-mediated hepcidin suppression is Epo-dependent. Hepatic Vhl and hepcidin mRNA levels in control, Vhl/Epo^{-/-} and Vhl/Epo^{-/-} mice treated with recombinant human EPO (rhEPO) (n=6, 6 and 5 respectively) are displayed. The right panel shows Hif-1α and Hif-2α protein levels in Vhl/Epo^{-/-} livers. Ponceau staining is used to assess for equal protein loading.

FIG. 10 provides Epo levels in control, Vhl/Epo^{-/-} and Vhl^{-/-} livers and kidneys (n=6, 6 and 3 respectively). Bottom panel shows serum Epo (sEpo) concentrations in control, Vhl/Epo^{-/-} and Vhl/Epo^{-/-} mice treated with recombinant human EPO (n=10, 6 and 4 respectively).

FIG. 11 shows hematocrit and reticulocyte counts in control, Vhl/Epo^{-/-} and rhEPO-treated Vhl/Epo^{-/-} mice and representative FACS analysis plot of CD71/Ter119 stained bone marrow (BM) and spleen cells from one control and one mutant mouse. Percentages of CD71^{high}/Ter119^{high}-positive cells are indicated in the right upper quadrant.

FIG. 12 shows liver (n=8, 4 and 5 respectively) and serum iron concentrations (n=10, 6 and 4 respectively) in control, Vhl/Epo^{-/-} and Vhl/Epo^{-/-} mice treated with recombinant human EPO, and H-ferritin protein levels in control and Vhl/Epo^{-/-} livers. β-actin served as loading control. Shown are mean values×SEM, *P<0.05; **P<0.01 and ***P<0.001 for comparisons of mutants to controls. Abb.: Co, Cre-negative littermate control; Hct, hematocrit; retic, reticulocytes; rhEPO, human recombinant EPO; Vhl/Epo^{-/-} (rhEPO), Vhl/Epo double mutant mice treated with recombinant human EPO.

FIG. 13 shows Hif-associated hepcidin suppression requires erythropoietic activity. FIG. 13 displays renal and hepatic Epo (n=3, 5 and 3 respectively) in control, Vhl/^{-/-} mutants with or without carboplatin treatment and liver hepcidin RNA levels (n=6, 4 and 3 respectively) in non-treated control, Cp-treated control and Cp-treated Vhl^{-/-} mutants. Lower panels show hematocrit, reticulocyte counts (n=3 and 4 respectively), serum Epo (sEpo) (n=3 and 5 respectively) and spleen to body weight ratios in non-treated control and Cp-treated Vhl^{-/-} mice (n=3 each).

FIG. 14 provides hepcidin mRNA levels in control and Hif-2α/Pax3-cre (P3) mutants exposed to chronic hypoxia (10% O₂ for 10 days) (n=3 each), and in thalassemic mice (th3/th3) and control littermates (+/+) (n=4 each). Shown are mean values±SEM, * P<0.05; **P<0.01 and ***P<0.001 for comparisons to control group or comparison to normoxia. Abb.: Co, Cre-negative littermate control; Cp, mice pre-treated with carboplatin; Hct, hematocrit; Hx, treatment with 10% O₂ for 10 days; retic, reticulocytes.

FIG. 15 shows that the elevation of serum Gdf15 in Vhl^{-/-} mice is Epo-dependent. Gdf15 mRNA levels in total spleen and bone marrow cell isolates and corresponding serum Gdf15 levels in pg/ml. Left panels, Vhl^{-/-} mutants and Cre⁻ littermate controls (n=3 and 4 respectively for mRNA analysis, for serum analysis n=4 each); middle panels, Vhl/Epo^{-/-} mice and Cre⁻ littermates controls (n=4 each); right panels, WT mice treated with recombinant human erythropoietin (rhEPO) or with vehicle (for mRNA analysis n=3 and 4 respectively, for serum analysis n=6 and 8 respectively). Shown are mean values±SEM, *P<0.05; **P<0.01 and ***P<0.001 for comparisons of mutants to controls.

FIG. 16 provides a schematic depiction of Hif's role in the regulation of hepcidin transcription in hepatocytes. Abb.: Co, Cre-negative littermate control mice or vehicle-treated WT mice; rhEPO, recombinant human EPO.

FIG. 17 is a characterization of Vhl^{-/-} mice. The left panel shows a schematic outline of the tamoxifen treatment schedule used to induce recombination. Arrows indicate on which days tamoxifen was injected. * indicates time point of analysis. Mutant mice were euthanatized for phenotyping on day 8. The right panel shows recombination analysis of the Vhl gene locus in control (Co) and Vhl^{-/-} tissues by genomic PCR on day 8. 1-lox represents the recombined allele, 2-lox indicates the non-recombined conditional allele.

FIG. 18 shows that complete blood counts were performed prior to tamoxifen injection on day 0 and on day 8. Shown are mean hematocrit (Hct), hemoglobin (Hb), rbc numbers, mean corpuscular volume (MCV) and reticulocyte counts (Retic) at day 0 and at day 8.

FIG. 19 provides mRNA levels of Dmt1 and Trfc in Vhl^{-/-} livers.

FIG. 20 shows liver, kidney and spleen to body weight ratios in control and Vhl^{-/-} mice at day 8 (n=4 and 3 respectively).

FIG. 21 provides the fraction (%) of CD71^{high}/Ter119^{high}-positive cells in bone marrow (BM) and spleen (n=3 each). Shown are arithmetic mean values±SEM, *P<0.05; **P<0.01 and ***P<0.001 for comparisons of mutants to controls. Abb.: Dmt1, divalent metal transporter 1; Trfc, transferrin receptor 1.

FIG. 22 presents a characterization of Vhl/Epo−/− mice. The left panel shows recombination analysis of the Epo gene locus in control (Co) and Vhl/Epo_{-/-} kidneys and livers by genomic PCR on day 8. The right panel shows recombination analysis of the Vhl gene locus in the same mice. Shown are two representative control and mutant mice. 1-lox indicates (A) Left panel shows recombination analysis of the Epo gene locus in control (Co) and Vhl/Epo_{-/-} kidneys and livers by genomic PCR on day 8. Right panel shows recombination analysis of the Vhl gene locus the recombined allele, 2-lox represents the non-recombined conditional allele.

FIG. 23 is a table that shows hematocrit (Hct), hemoglobin (Hb), rbc numbers, mean corpuscular volume (MCV) and reticulocyte counts (retic) at day 0 and day 8.

FIG. 24 provides Vegf and Dmt1 mRNA levels in control and Vhl/Epo^{-/-} livers (n=6 each).

FIG. 25 shows the fraction (%) of CD7lhigh/Ter119high-positive cells in bone marrow (BM) and spleen from control, Vhl/Epo^{-/-} and Vhl/Epo^{-/-} mice treated with recombinant human EPO (n=10, 6 and 5 respectively). Shown are arithmetic mean values±SEM, *P<0.05; **P<0.01 and ***P<0.001 for comparisons of mutants to controls. Abb.: Dmt1, divalent metal transporter 1; rhEPO, human recombinant EPO; Vhl/Epo^{-/-} (rhEPO), Vhl/Epo double mutant mice treated with recombinant human EPO.

FIG. 26 illustrates that Gdf15 suppresses hepcidin in Hep3B cells. Indeed, it shows Gdf15 mRNA levels in Ter119-positive (+) and Ter119-negative (−) spleen and bone marrow (BM)-derived cells from Vhl^{-/-} and control mice (Co), enriched with immunomagnetic beads. Notably, while Gdf15 mRNA levels were increased in Vhl-deficient Ter119-enriched splenic cell preparations compared to control, higher levels of Gdf15 message were detected in splenic cells that did not bind to Ter119 magnetic beads. It is therefore possible that most of splenic Gdf15 is either of non-erythroid origin or is produced by Ter119^{low} erythroid progenitor cells that do not efficiently bind to Ter119 magnetic beads.

FIG. 27 shows Twsg1 mRNA levels in total spleen and BM cell isolates. In the left panel are Vhl^{-/-} mutants and Cre− littermate controls (n=4 each). In the middle panel are Vhl/Epo^{-/-} mice and Cre− littermate controls (n=4 each). And in the right panel are WT mice treated with recombinant human erythropoietin (rhEPO) or with vehicle (n=3 and 4 respectively).

FIG. 28 provides a real-time PCR analysis of HAMP levels in vehicle- or Gdf15-in the same mice as in FIG. 26 and FIG. 27. Shown are two representative control and mutant mice. 1-lox indicates treated (750 pg/ml) Hep3B cells (shown are the means of 3 independent experiments).

FIG. 29 presents a real-time PCR analysis of Tmprss6 and furin mRNA levels in Vhl−/− and control mice (n=3 and 4 respectively). Shown are mean values±SEM, **P<0.01 and ***P<0.001 for comparisons of mutants to controls. Abb.: Gdf15, growth differentiation factor 15; Tmprss6, transmembrane protease serine 6/matriptase-2; Twsg1, twisted gastrulation homolog 1.

FIG. 30 provides treatment with recombinant EPO results in serum Gdf15 elevation in wild type mice. Serum Gdf15 levels are shown in pg/ml.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods and kits for evaluating and/or monitoring a treatment program, such as in an anemic subject and/or in a subject receiving treatment for anemia, including determining a presence or an amount in a biological sample from the subject of growth differentiation factor 15 (“GDF 15” or “Gdf 15,” which are used interchangeably to refer to the polypeptide in the subject of interest).

The presently-disclosed subject matter is based, in part, on the discovery of serum Gdf15 levels being responsive to the administration of recombinant EPO, and on the discovery that the increased Gdf15 levels are sufficient to suppress hepcidin (See Examples, FIG. 15, and FIG. 29).

The presently-disclosed subject matter includes a method for evaluating and/or monitoring a treatment program for a subject, which involves providing a biological sample from the subject; determining a presence or an amount of growth differentiation factor 15 (GDF15) in the sample and/or determining a presence or an amount of hepcidin in the sample; and comparing the presence or the amount of the GDF15 and/or hepcidin to a reference, wherein the treatment program is evaluated based on a measurable difference in the presence or the amount of the GDF15 and/or hepcidin as compared to the reference.

In some embodiments of the presently-disclosed subject matter, the subject is receiving treatment with recombinant erythropoietin (EPO), a HIF-stabilizing composition, and/or a composition that stimulates endogenous EPO synthesis. In some embodiments, the subject is anemic and/or is receiving treatment for anemia. In some embodiments, the subject is receiving dialysis. In some embodiments, the subject is receiving renal dialysis, and the subject is resistant to treatment with recombinant EPO and/or is not responding to treatment with recombinant EPO.

By way of providing non-limiting examples, the presently-disclosed subject matter could be applied in the following manner. The subject could be a human dialysis patient who requires high doses of intravenous or subcutaneous EPO administration to maintain red blood cell count in a certain target range. If the subject's red blood cell count numbers are falling, iron saturation is borderline despite repeated iron therapy. A medical care professional needs to decide whether to give additional iron and/or whether to increase EPO dosing. Determining a presence or an amount of GDF15 in a biological sample (or in serial samples) can be used to assist in the identification of the subject as a likely responder or non-responder to additional iron administration and/or increased EPO doses. In some embodiments GDF15/hepcidin ratios (of amounts in a biological sample) (or in serial samples) can be used to assist in the identification of the subject as a likely responder or non-responder to additional iron administration and/or increased EPO doses.

By way of providing non-limiting examples, the presently-disclosed subject matter could be used in the monitoring of bone marrow/erythroid responses/prediction of bone marrow responses to any kind of pharmacologic intervention that aims at increasing erythropoiesis (e.g. HIF stabilizers or others) or that aim at improving iron utilization by the bone marrow or correction of functional iron-deficiency anemia.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As will be recognized by one of ordinary skill in the art, the term “measurable difference” may refer to any increase or decrease in a value, quantity, amount and/or measure relative to a control and/or reference amount. The “measurable difference” of the present disclosure can be, for example, about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% increase or decrease in a quantity and/or amount of a component relative to a reference amount of that component.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The terms “subject” or “subject in need thereof” refer to a target of administration, which optionally displays symptoms related to a particular disease, condition, disorder, or the like. The subject(s) of the herein disclosed methods can be human or non-human (e.g., primate, horse, pig, rabbit, dog, sheep, goat, cow, cat, guinea pig, rodent, and non-mammals). The term “subject” does not denote a particular age or sex. Thus, adult and newborn subjects, as well as fetuses, whether male or female, are intended to be covered. The term “subject” includes human and veterinary subjects.

The terms “treatment” or “treating” refer to the medical management of a subject with the intent to cure, ameliorate, stabilize, or prevent a condition or disorder. This term includes active treatment, that is, treatment directed specifically toward the improvement of a condition, and also includes causal treatment, that is, treatment directed toward removal of the cause of the associated condition. In addition, this term includes palliative treatment, that is, treatment designed for the relief of symptoms rather than the curing of the condition; preventative treatment, that is, treatment directed to minimizing or partially or completely inhibiting the development of symptoms or disorders of the associated condition; and supportive treatment, that is, treatment employed to supplement another specific therapy directed toward the improvement of the associated disease, pathological condition, or disorder.

With regard to administering the compound, the term “administering” refers to any method of providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, intracameral (into anterior chamber) administration, subretinal administration, sub-Tenon's administration, peribulbar administration, administration via topical eye drops, and the like. Administration can be continuous or intermittent. In various aspects, a preparation can be administered therapeutically; that is, administered to treat an existing disease or condition (e.g., exposure to OP compounds). In further various aspects, a preparation can be administered prophylactically; that is, administered for prevention of a disease or condition.

As used herein, the phrase a “disorder of iron homeostasis” and/or the phrase “a disorder of iron metabolism” may be used interchangeably and may include, for example, an anemia, a disorder of iron metabolism, a sideroblastic anemia, a hypochromic microcytic anemia, hereditary hemochromatosis, a congenital anemia, and/or the like.

In some embodiments, the present disclosure provides methods for treating a disorder of iron homeostasis in a subject, comprising the steps of (i) providing a biological sample from the subject; (ii) determining an amount of growth differentiation factor 15 (GDF15) in the sample; (iii) comparing the amount of the GDF15 to a reference amount of GDF15; and/or (iv) administering to the subject an effective amount of at least one of iron and erythropoietin (EPO) if there is a measurable difference in the amount of GDF15 in the sample as compared to the reference amount of GDF15.

In some embodiments, the subject is a human, and in certain embodiments, the sample comprises a blood sample and/or a serum sample. Further, in some embodiments, the effective amount of iron and/or EPO may be administered to the subject on an hourly, daily and/or weekly basis. And in some embodiments, the administration interval is determined by a medical professional.

In some embodiments, a disorder of iron homeostasis comprises anemia. And some methods of the present disclosure may include a step of monitoring a bone marrow and/or an erythroid response in the subject and/or a step of determining a red blood cell count in a sample obtained from the subject.

Moreover, in certain embodiments of the present disclosure, the subject may be receiving dialysis, may be resistant to treatment with recombinant EPO and/or may not be responding to treatment with recombinant EPO, and/or may be receiving treatment with recombinant erythropoietin, a hypoxia-inducible factor (HIF)-stabilizing composition, and/or a composition that stimulates endogenous EPO synthesis. And in some embodiments, the method(s) of the present disclosure may further comprise the steps of: (i) determining an amount of hepcidin in a sample; (ii) comparing the amount of hepcidin in the sample to a reference amount of hepcidin; and/or (iii) determining a ratio of the amount of GDF15 in the sample to the amount of hepcidin in the sample.

The present disclosure additionally provides, in certain embodiments, a method for treating disorder of iron homeostasis in a subject, the method comprising: (i) providing a biological sample from the subject; (ii) determining an amount of hepcidin in the sample; (iii) comparing the amount of the hepcidin to a reference amount of hepcidin; and/or (iv) administering to the subject an effective amount of at least one of iron and erythropoietin (EPO) if there is a measurable difference in the amount of hepcidin in the sample as compared to the reference amount of hepcidin. In some embodiments, the effective amount of at least one of iron and EPO is administered to the subject on an hourly, daily and/or weekly basis. And in a particular embodiment, the disorder of iron homeostasis comprises anemia.

Meanwhile, in some embodiments, the present disclosure provides a method for evaluating and/or monitoring a treatment program for a subject, the method comprising: (i) providing a biological sample from the subject; (ii) determining a presence or an amount of GDF15 in the sample and/or determining a presence or an amount of hepcidin in the sample; and/or (iii) comparing the presence or the amount of the GDF15 and/or hepcidin to a reference, wherein the treatment program is evaluated based on a measurable difference in the presence or the amount of the GDF15 and/or hepcidin as compared to the reference. In certain embodiments, the subject is: (i) receiving treatment with recombinant EPO, a HIF-stabilizing composition, and/or a composition that stimulates endogenous EPO synthesis; (ii) anemic and/or receiving treatment for anemia; (iii) receiving dialysis; (iv) receiving renal dialysis; (v) resistant to treatment with recombinant EPO; and/or (vi) not responding to treatment with recombinant EPO. Additionally, in some embodiments, the methods of the present disclosure comprise a step of: (i) determining a ratio of an amount of GDF15 to an amount of hepcidin in a sample; (ii) monitoring at least one bone marrow and/or erythroid response in a subject; and/or (iii) predicting a bone marrow response to a treatment program, such as a pharmacologic treatment program designed to improve iron utilization by bone marrow or correction of functional iron-deficiency anemia.

And in some embodiments, the reference comprises (i) a control and/or (ii) a level of the GDF15 and/or hepcidin in a sample from a subject taken over a time course and/or at pre-determined intervals of time. In further embodiments, the reference comprises a sample from the subject collected prior to initiation of a treatment program. And in some embodiments, the biological sample is collected after initiation of the treatment program. In certain embodiments, the reference comprises a standard sample. And in some embodiments, the reference comprises control data. In still further embodiments, the reference comprises a level of the GDF15 and/or hepcidin in one or more samples from one or more individuals who are known responders or who are known non-responders to treatment with recombinant erythropoietin, a HIF-stabilizing composition and/or a composition that stimulates endogenous EPO synthesis.

In certain embodiments, the methods of the present disclosure include a step of alternating a treatment and/or alternating a treatment program. In some embodiments, alternating a treatment program involves administering iron and/or EPO to a subject and/or altering the dose of iron and/or EPO being administered to the subject. Certain embodiments of the methods of the present disclosure may be performed and/or carried out in vitro.

The presently-disclosed subject matter is further illustrated by the following specific but non-limiting examples. The following examples may include compilations of data that are representative of data gathered at various times during the course of development and experimentation related to the present invention(s).

### EXAMPLES

A genetic approach was used to disengage HIF activation from EPO synthesis, and it was found that HIF-mediated suppression of hepcidin required EPO inducibility, which was associated with increased erythropoietic activity and elevated serum levels of growth differentiation factor 15. However, when erythropoiesis was inhibited pharmacologically, hepcidin was no longer suppressed despite profound elevations in serum EPO, indicating that EPO by itself is not directly involved in hepcidin regulation. Taken together, in vivo evidence is provided that shows hepcidin suppression by the HIF pathway occurs indirectly through stimulation of EPO-induced erythropoiesis.

The increased production of red blood cells (rbc) and thus increased 0₂-carrying capacity of blood represents a major adaptation to systemic hypoxia. This important physiologic response consists of cell type-specific changes that include increased EPO production in kidney and liver, enhanced iron uptake and utilization, as well as adjustments in the bone marrow microenvironment that facilitate erythroid progenitor maturation and proliferation (1). Hepcidin, encoded by the HAMP gene, is a hypoxia-regulated, small polypeptide produced in hepatocytes, which in its processed form consists of 25 amino acids and plays a central role in the maintenance of systemic iron homeostasis. It suppresses intestinal iron uptake and release from internal stores by facilitating the degradation and internalization of the only known iron exporter, ferroportin, which is expressed on the surface of enterocytes, hepatocytes and macrophages. Chronic elevation of serum hepcidin, which often associates with inflammatory states, reduces ferroportin surface expression and produces hypoferremia. In contrast, constitutively low hepcidin production in the liver, e.g. due to genetic defects in intracellular signaling pathways that control hepcidin transcription, results in persistent hyperferremia and the development of hemochromatosis (2).

Central mediators of hypoxia-induced erythropoiesis are the O₂-regulated basic helix-loop-helix transcription factors HIF-1 and HIF-2. They consist of an O₂-sensitive alpha-subunit (HIF-1α and HIF-2α, which is also known as endothelial PAS domain protein 1, EPAS1) and a constitutively expressed beta-subunit, HIF-β, which is also known as the aryl-hydrocarbon receptor nuclear translocator (ARNT). In vivo studies have identified HIF-2 as the main regulator of EPO (1), the glycoprotein that prevents apoptosis of erythroid progenitor cells and is essential for the maintenance of normal erythropoiesis and the increase in rbc production under hypoxia (3). The activity of HIF is controlled by O₂-, iron- and ascorbate-dependent dioxygenases, also known as prolyl-hydroxylase domain-containing proteins 1-3 (PHD1-3), which use 2-oxoglutarate as substrate for the hydroxylation of specific proline residues in HIF-α. Hydroxylation of HIF-α permits binding to the von Hippel-Lindau (VHL)-E3 ubiquitin ligase complex, which results in proteasomal degradation of HIF-α (4).

Experimental studies in cell culture and in animals, as well as clinical data from patients with Chuvash polycythemia, who are homozygous for the VHL R200W mutation, support the notion that hepcidin synthesis involves the VHL/HIF/PHD axis (5-8). However, the molecular basis of its O₂-dependence, in particular the role of HIF in its regulation is unclear. Genetic studies with iron-deficient mice in conjunction with transcriptional assays have suggested that HIF-1 activation in hepatocytes suppresses hepcidin directly via hypoxia response element (HRE)-dependent mechanisms (8). However, this model is debated and more recent in vitro experiments suggest that HIF does not function as a direct transcriptional repressor of hepcidin (9, 10).

A second model of hypoxia-induced hepcidin suppression involves iron-dependent signaling pathways that control hepcidin transcription. Signaling through either HFE, which is mutated in patients with hereditary hemochromatosis, transferrin receptor 1 (TRFC) and transferrin receptor 2 (TFR2) (2, 11, 12), or hemojuvelin (HJV), which acts as a co-receptor for bone morphogenetic protein 6 (BMP6) increases hepcidin transcription in a SMAD-dependent fashion (13-15). In vitro studies have shown that HIF induces furin, a proprotein convertase that cleaves HJV and generates soluble HJV, which in turn suppresses hepcidin by competing for BMP6, thereby antagonizing signaling through membrane-bound HJV (16, 17). Similarly, transmembrane protease serine 6 (TMPRSS6), also known as matriptase-2, has been identified as HIF-regulated and is predicted to blunt BMP6/HJV-mediated signals under hypoxia (18-20). A direct effect of EPO on hepcidin transcription has also been postulated. Studies with primary mouse hepatocytes and HepG2 cells have shown that EPO, in a dose-dependent fashion, is capable of regulating hepcidin transcription via EPO receptor (EPOR) and CCAAT/enhancer-binding protein (C/EBP) α activation (21).

Another model proposes that stimulation of erythropoiesis generates a bone marrow-derived signal which suppresses hepcidin in the liver (22). Growth differentiation factor 15 (GDF15), an iron- and O₂-regulated (HIF-independent) member of the TGF-β superfamily, is secreted from maturing erythroblasts and suppresses hepcidin transcription in primary human hepatocytes and hepatoma cells (23, 24). Since very high levels of serum GDF15 were found in patients with α- and β-thalassemia, it was proposed that GDF15 is the bone marrow-derived factor that suppresses hepcidin under conditions of stimulated erythropoiesis (24). While high serum GDF15 levels were found in patients with syndromes of ineffective erythropoiesis (24-27), the association between serum GDF15 and serum hepcidin levels in other forms of anemia was less evident. This raised the possibility that GDF15 may be a marker of ineffective or apoptotic erythropoiesis. Nevertheless, its role in hepcidin regulation under physiologic or other pathologic conditions remains to be elucidated (for a recent review on this topic see (28)).

To specifically dissect the role of the VHL/HIF/PHD axis and EPO in the hypoxic suppression of hepcidin in vivo, a genetic approach was used to disengage Hif activation from Epo synthesis in mice. Tamoxifen-inducible Cre/loxP-mediated recombination was utilized to activate Hif-1 and Hif-2 via ablation of Vhl while simultaneously inactivating Epo. It was found that hypoxia/Hif-mediated suppression of hepcidin required Epo inducibility and was associated with elevated serum Gdf15 levels. Including additional genetic models, it is demonstrated that increased erythropoietic drive is required for hepcidin suppression under conditions of hepatic Hif activation irrespective of serum Epo levels. The genetic data establish that Hif activation in hepatocytes suppresses hepcidin indirectly through Epo-mediated stimulation of erythropoiesis.

Conditional Inactivation of Vhl Results in Hif-Dependent Hepcidin Suppression.

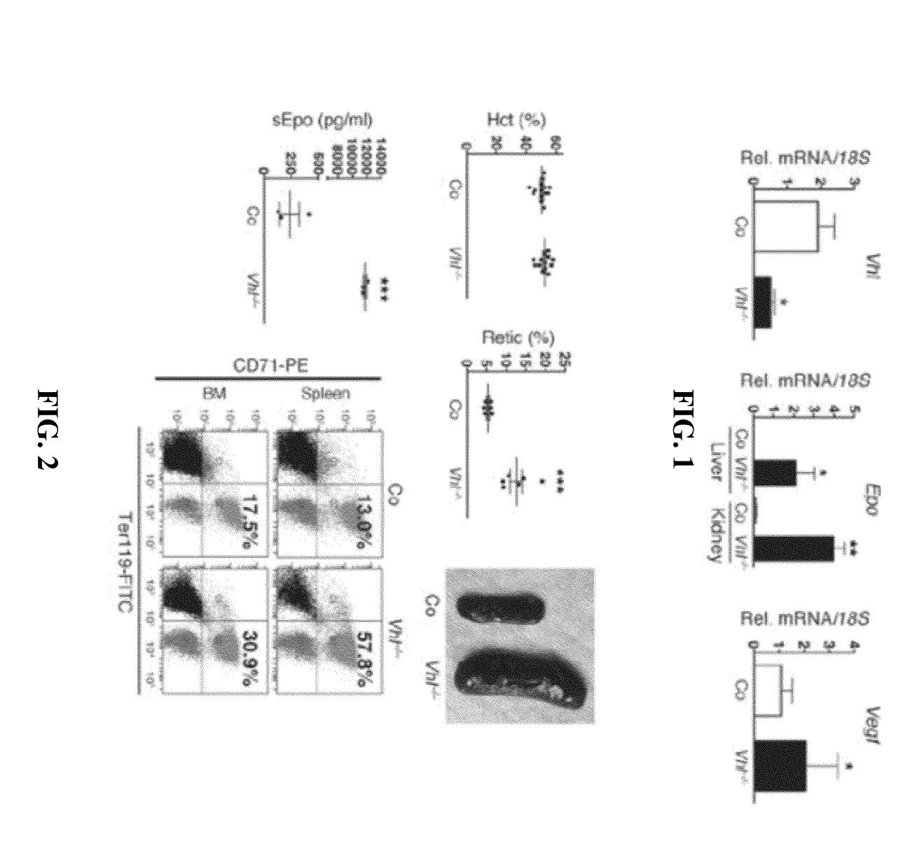

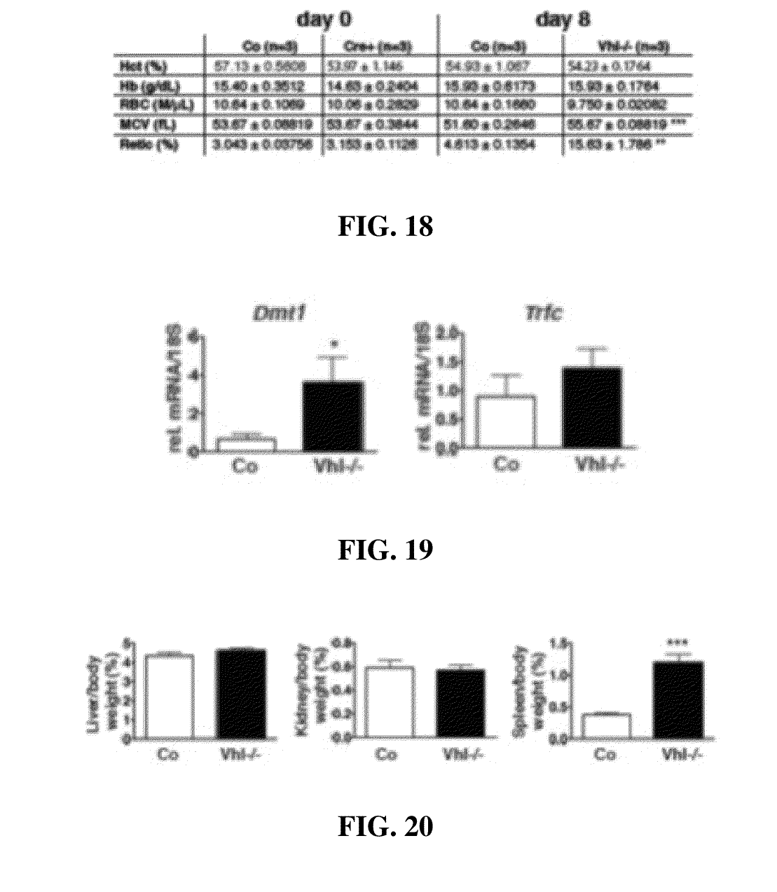

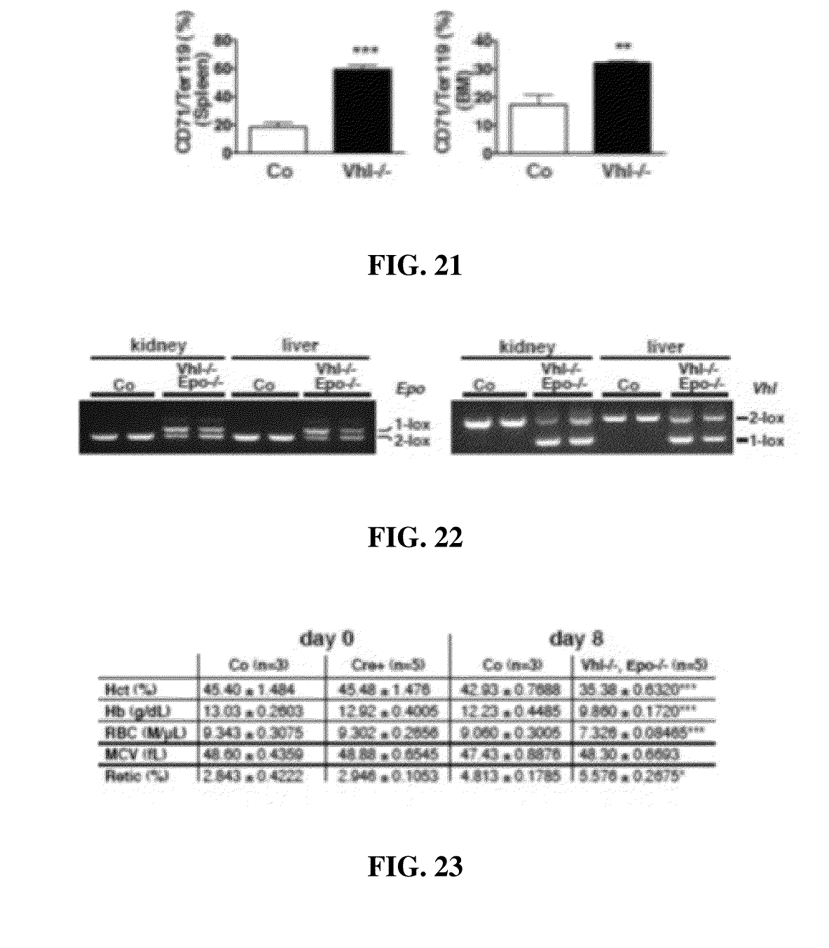

For the genetic dissection of hepcidin regulation by the HIF oxygen-sensing pathway, a model of acute pVHL inactivation was established using a globally expressed tamoxifen-inducible Cre-recombinase under the control of the ubiquitin c promoter, Ubc-cre/ERT2 (29). Although hepatocyte-specific Vhl inactivation via Cre-recombinase driven by the albumin promoter suppresses hepatic hepcidin (Hamp1) (8, 30), constitutive Hif activation in the liver has profound effects on glucose and fatty acid metabolism and results in sick animals that die from liver failure between the ages of 6 and 12 weeks (31), which makes the interpretation of hematologic data difficult and limits experimental options. To achieve efficient recombination in Ubc-cre/ERT2 mice that were homozygous for the Vhl floxed allele, 4 doses of tamoxifen were administered over a period of 7 days, followed by phenotypic analysis on day 8 (experimental time line is shown in FIG. 17-21). Tamoxifen administration resulted in an ˜75% reduction in hepatic Vhl mRNA levels, stabilization of both Hif-1α and Hif-2α homologs and increased expression of Hif target genes, such as Epo, Vegf and divalent metal transporter 1 (Dmt1) (FIG. 1 and FIG. 17-21). Recombination analysis by genomic PCR indicated efficient recombination in the kidney and in other organs (FIG. 17-21 and data not shown). Since hepatic and renal Epo synthesis was stimulated in Vhl^{-/-} mutant mice, serum Epo levels were elevated to 11733±217.0 pg/ml compared to Cre⁻ control mice (235.3±89.8 pg/ml). This resulted in increased formation of CD71^{high}/Ter119^{high}-positive erythroblasts in spleen and bone marrow (in spleen 59.33±2.93% for mutants vs. 18.2±3.54% for control mice, and 32.2±0.723% vs. 17.33±3.48% in bone marrow; n=3 each), splenomegaly and reticulocytosis (12.64±1.57% for mutants vs. 5.37±0.35% for controls, n=6 each), all of which is consistent with increased erythropoietic activity (FIG. 2 and FIG. 17-21).

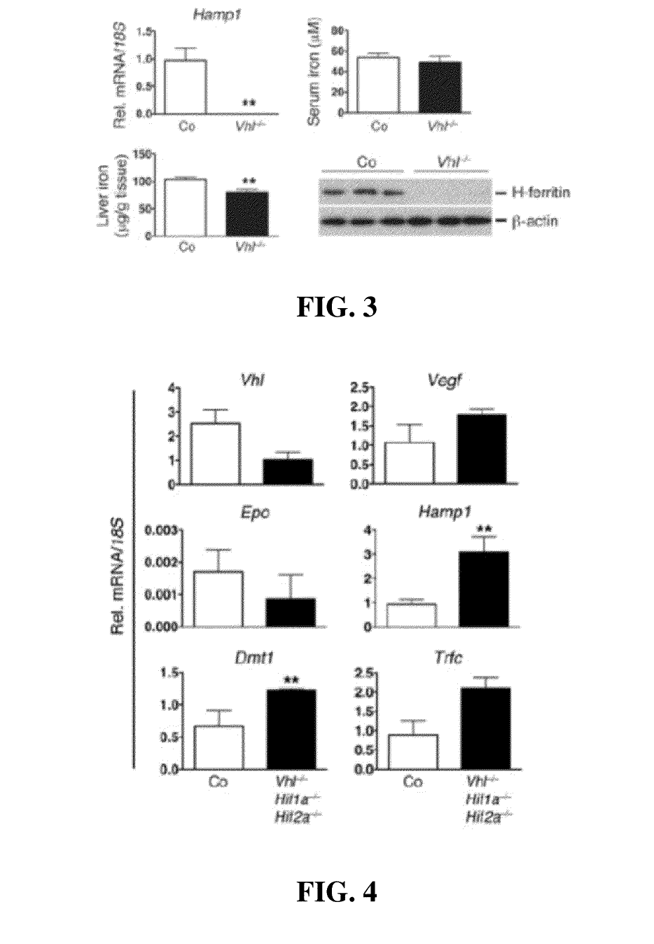

Hematocrit (Hct), hemoglobin (Hb) and rbc values in Vhl^{-/-} mutants were not different from controls at day 8 after the first tamoxifen injection and were found to be increased at day 16; Hb values increased from 14.13±0.186 g/dL in controls to 16.07±0.73 g/dL in mutants on day 16, n=3 each (FIG. 17-21 and data not shown). Hepcidin mRNA levels (reported here are data for Hamp1) were undetectable in Vhl^{-/-} livers by real time PCR analysis (FIG. 3). While serum iron levels did not differ between mutants and Cre⁻ controls, iron was decreased in Vhl^{-/-} livers, which was associated with reduced ferritin heavy chain-1 (H-ferritin) levels (FIG. 3). This is expected as the ferritin 5′-UTR contains an iron response element (IRE) that mediates translational inhibition in the presence of low intracellular iron. Taken together, these results demonstrate that acute global inactivation of Vhl results in increased erythropoietic activity and associates with decreased hepcidin expression in the liver.

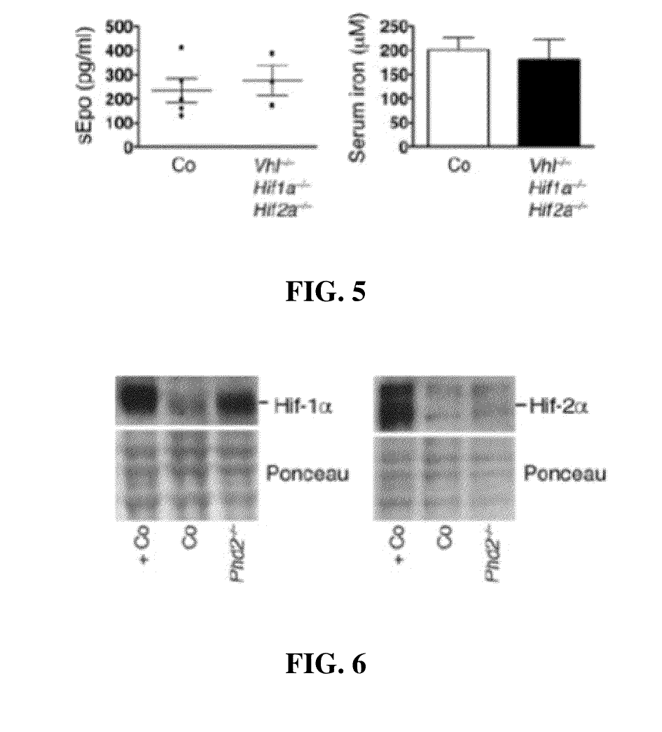

To investigate the role of Hif in VHL-associated hepcidin regulation, mice that permitted global inactivation of both Hif-1α and Hif-2α in a Vhl-deficient background were generated. Epo or Vegf were not significantly induced or decreased in Vhl/Hif-1/Hif-2^{-/-} livers compared to Cre− littermate control mice (FIG. 4), which suggests a) that their increased expression in Vhl^{-/-} livers is Hif-dependent and b) that Hif-1 and Hif-2 do not participate in their transcriptional regulation under baseline, i.e. normoxic conditions. This is consistent with the inventors' previous studies in hepatocyte-specific Vhl knock out mice, where Hif-2 was identified as the main regulator of hepatic Epo synthesis. With these studies it was demonstrated that inactivation of both Hif-1α and Hif-2α in a Vhl−/− background completely abrogated Epo induction (32). Consequently, serum Epo concentrations in Vhl/Hif-1/Hif-2^{-/-} mice did not significantly differ from Cre⁻ control mice (277.5±61.66 pg/ml in mutants vs. 235.1±50.69 pg/ml in controls; n=3 and n=5 respectively) (FIG. 5). The abrogation of Epo induction in triple mutants was associated with a statistically significant increase in hepcidin mRNA levels in Vhl/Hif-1/Hif-2^{-/-} livers compared to Cre⁻ controls; P=0.0035 for n=3. In summary the data establish that the suppression of hepcidin in Vhl-defective livers requires Hif stabilization.

Hif-1 Does Not Suppress Hepcidin in Phd2−/− Livers.

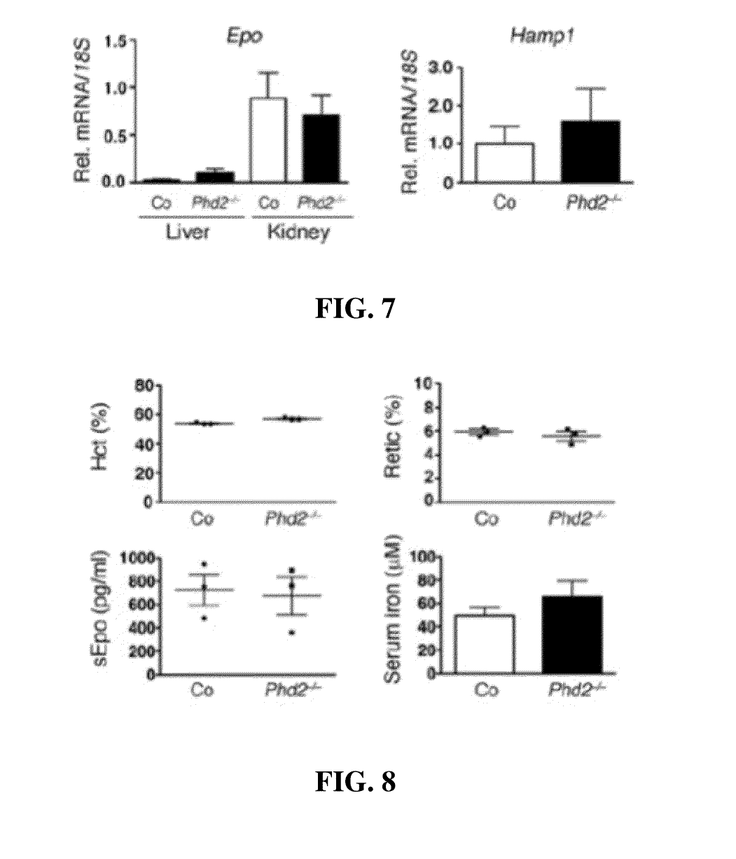

Since hepatic Hif-1 has been shown to participate in the regulation of hepcidin in iron deficiency anemia (8), a mouse model, which specifically activates hepatic Hif-1 in a genetic background that is wild type for Vhl, was used. Mice with hepatocyte-specific inactivation of Phd2 were generated. Phd2, also known as EglN1, is the major Hif prolyl-4-hydroxylase that targets Hif-α subunits for hydroxylation and subsequent proteasomal degradation under normoxia. Phd2 inactivation in hepatocytes (EglN1^{2lox/2lox}; Albumin-cre) resulted in stabilization of Hif-1α, but not of Hif-2α (FIG. 6), which is consistent with previous reports (33). Surprisingly, hepcidin expression levels in Phd2-deficient livers did not change compared to controls (FIG. 7). Also, hepatocyte-specific Phd2 inactivation did not increase Epo mRNA levels, nor did it stimulate erythropoietic bone marrow activity, as an increase in blood reticulocytes and Hct was not observed (reticulocyte count: 5.93±0.22% vs. 5.58±0.381%; Hct: 54.33±0.33% and 57.33±0.33% respectively, n=3 each) (FIG. 7 and FIG. 8). This is consistent with the inventors' previous observation that hepatic Epo synthesis is predominantly Hif-2-regulated (32). The inventors' findings indicate that Hif-1α stabilization alone is not sufficient to suppress hepcidin in hepatocytes. Furthermore, analysis of Phd2 knock out mice suggests that Hif-associated hepcidin suppression is linked to Hif-2-dependent stimulation of erythropoiesis.

Hif-Mediated Hepcidin Suppression Requires Epo Inducibility.

The findings in liver-specific Phd2 knock out mice, suggested that hepatic Hif-2 activation and/or Hif-2-stimulated erythropoiesis led to the suppression of hepcidin in Vhl-deficient livers. To determine whether Hif-induced hepcidin suppression in Vhl^{-/-} mice is dependent on the ability to synthesize Epo, the inventors of the present disclosure generated a genetic mouse model in which Hif activation can be dissociated from Epo synthesis and Epo-induced erythropoietic activity. In this model, both Hif-1 and Hif-2 are activated in hepatocytes and other cell types without any concomitant increase in renal and hepatic Epo production. For this purpose the inventors bred the Epo-21ox allele into the Vhl-2lox background, and generated mice that permitted global, tamoxifen-inducible and concurrent Vhl and Epo gene inactivation (Vhl^{2lox/2lox}; Epo^{2lox/2lox}; Ubc-cre/ERT2).

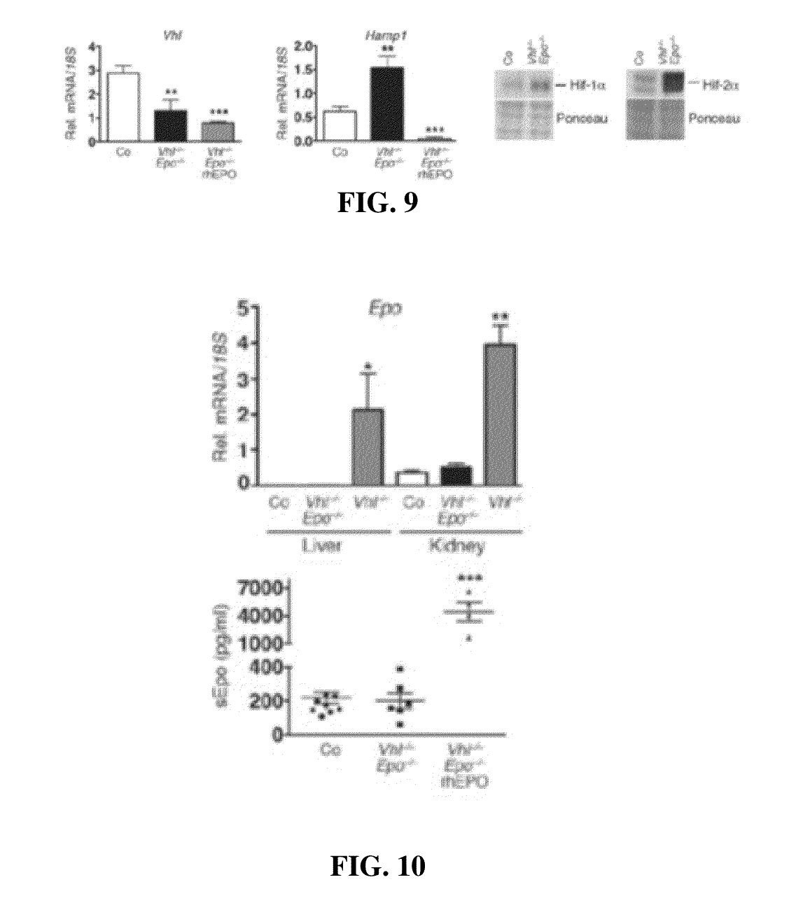

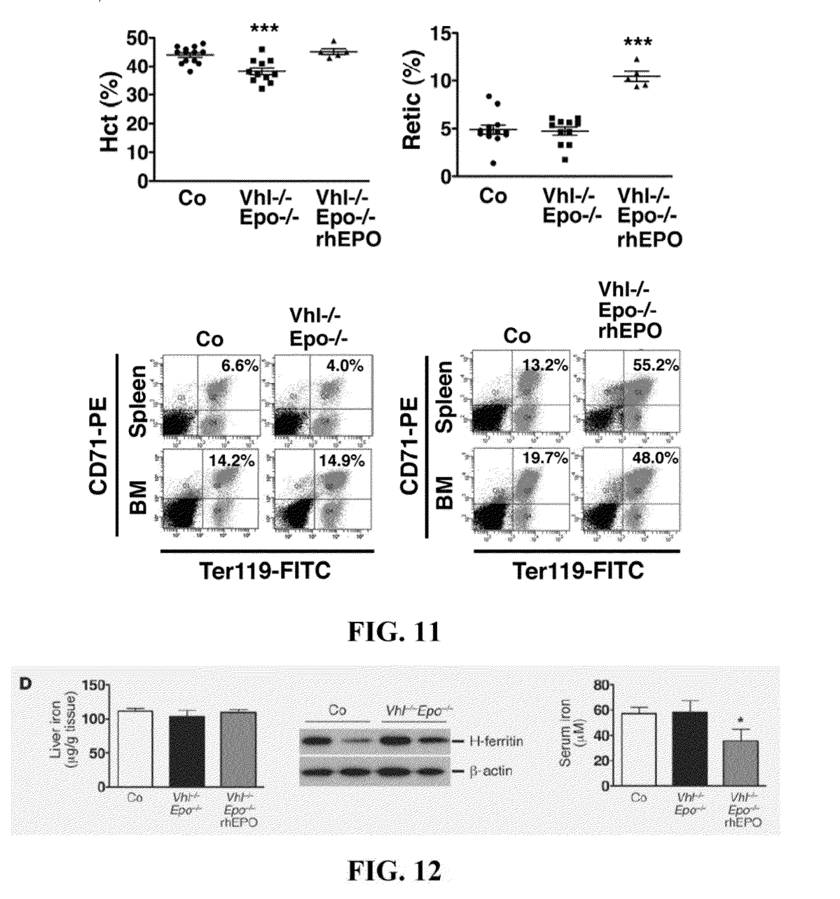

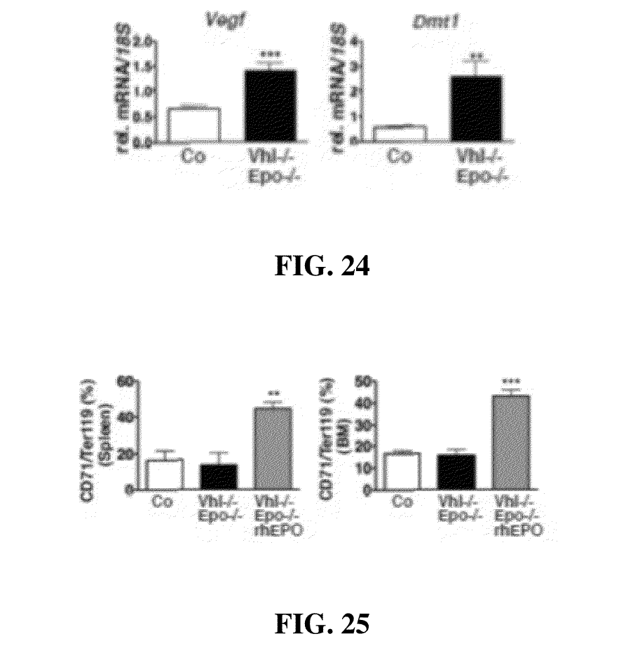

While Vhl ablation in Vhl/Epo^{-/-} mice resulted in stabilization of hepatic and renal Hif-1α and Hif-2α (FIG. 9), as well as increased Vegf and Dmt-1 mRNA levels (FIG. 24), Epo mRNA was not induced in the liver and kidney (FIG. 10, hepatic and renal Epo mRNA levels in Vhl−/− mice are shown for comparative purposes). Serum Epo levels in Vhl/Epo^{-/-} mice were slightly decreased (200.1±46.6 pg/ml vs. 217.3±33.4 pg/ml in control animals, n=6 and 10 respectively), but not significantly different from control mice, while rbc numbers, Hct and Hb values were decreased compared to controls (FIG. 11 and FIG. 22). Rbc numbers, Hct and Hb values decreased further over time; at day 16 after the first tamoxifen injection, mean Hct in Vhl/Epo^{-/-} mutants was 24.03±1.937%, mean Hb was 6.5±0.59 g/dL and mean rbc count was 5.01±0.41 M/μl (n=3), which is consistent with hypoproliferative anemia that develops in mice with global Epo inactivation (34). Despite Hif-1α and Hif-2α stabilization in the liver, hepcidin was no longer suppressed and increased significantly by approximately 3-fold; P=0.0032, n=6) (FIG. 9). These findings indicate that a) hepcidin is not directly regulated by either Hif-1 or Hif-2 and b) that its suppression is dependent on the induction of Epo synthesis, while acute Epo-deficiency increases its transcription.

Since Epo-dependence of VHL-associated hepcidin regulation was established, the inventors asked whether administration of recombinant EPO was able to overcome the genetic Epo deficiency and could restore hepcidin suppression in Vhl/Epo^{-/-} livers. For this, 200 IU of recombinant human EPO (rhEPO) were administered to Vhl/Epo^{-/-} mice i.p. every day for 3 days prior to mouse phenotyping. While a rise in rbc numbers was not seen 4 days after the initiation of rhEPO treatment (FIG. 11), reticulocyte counts increased from 4.91±0.61% to 10.44±0.53% (n=10 and 5 respectively), and CD71^{high}/Ter119^{high}-positive erythroblasts increased from 16.42±5.28% to 45±3.35% in spleen and from 16.5±1.25% to 43.36±2.78% in bone marrow (n=10 and 5 respectively) (FIG. 11 and FIG. 22-25). Restoration of erythropoietic activity suppressed hepcidin in Vhl/Epo^{-/-} livers (FIG. 9). Taken together, the data provide genetic evidence that hepcidin suppression in Vhl^{-/-} livers requires intact Epo synthesis and is not directly dependent on Hif-1 and/or Hif-2 activation in hepatocytes.

Hif-Mediated Hepcidin Suppression Requires Erythropoietic Activity and Associates with Increased Serum Gdf15 Levels.

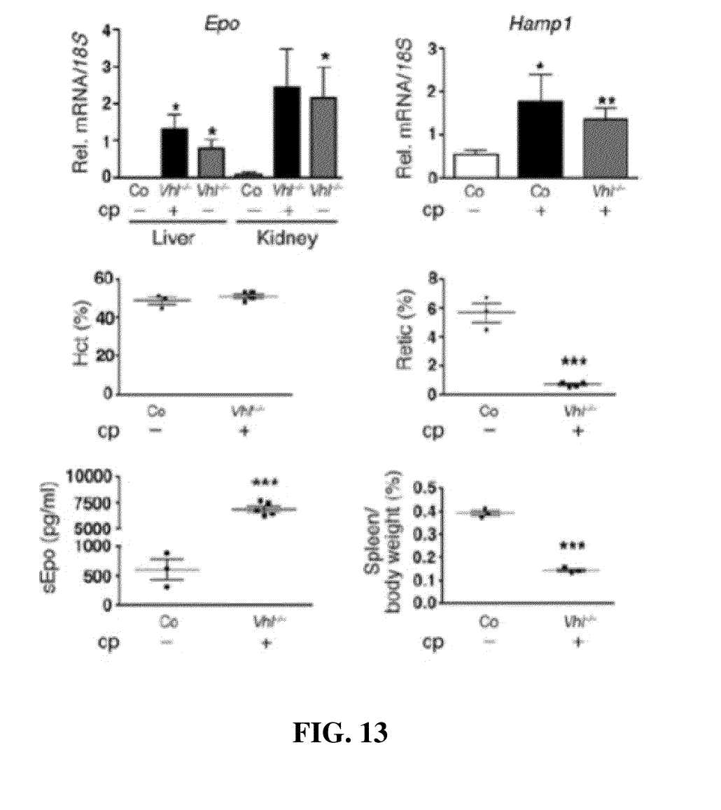

Although the genetic data established a clear role for Epo in the regulation of hepcidin, it was unclear whether Epo effects on Vhl^{-/-} hepatocytes were direct, e.g. via EpoR activation, or whether Hif-mediated hepcidin suppression was dependent on Epo-induced erythropoietic activity (22). To investigate the role of erythropoiesis in the regulation of hepcidin in this model, Vhl^{2lox/2lox}; Ubc-cre/ERT2 animals were pre-treated with carboplatin (Cp) to achieve efficient bone marrow suppression. Cp-treated and vehicle-treated mice were analyzed one day after the final tamoxifen injection (day 8). Comparable increases were found in hepatic and renal Epo mRNA levels in both Cp-treated and vehicle-treated Vhl^{-/-} mice, which suggested similar degrees of recombination in both groups (FIG. 13). Despite the presence of very high serum Epo levels (6911±276.9 pg/ml), reticulocyte counts were severely reduced in Cp-treated Vhl^{-/-} mice (0.66±0.08% in Cp-treated Vhl^{-/-} mice vs. 5.64 ±0.65% in vehicle-treated controls, n=4 and 3 respectively), which is consistent with robust inhibition of erythropoietic activity by Cp (FIG. 13). Most strikingly, the strong induction of renal and hepatic Epo synthesis in Cp-treated Vhl^{-/-} animals was not associated with hepcidin suppression, but with significantly increased hepcidin mRNA levels; P=0.0044 for n=3 and 6 for control (FIG. 13). Taken together, these findings indicate that Epo-dependent induction of erythropoiesis is required for the suppression of hepcidin in Vhl^{-/-} mice.

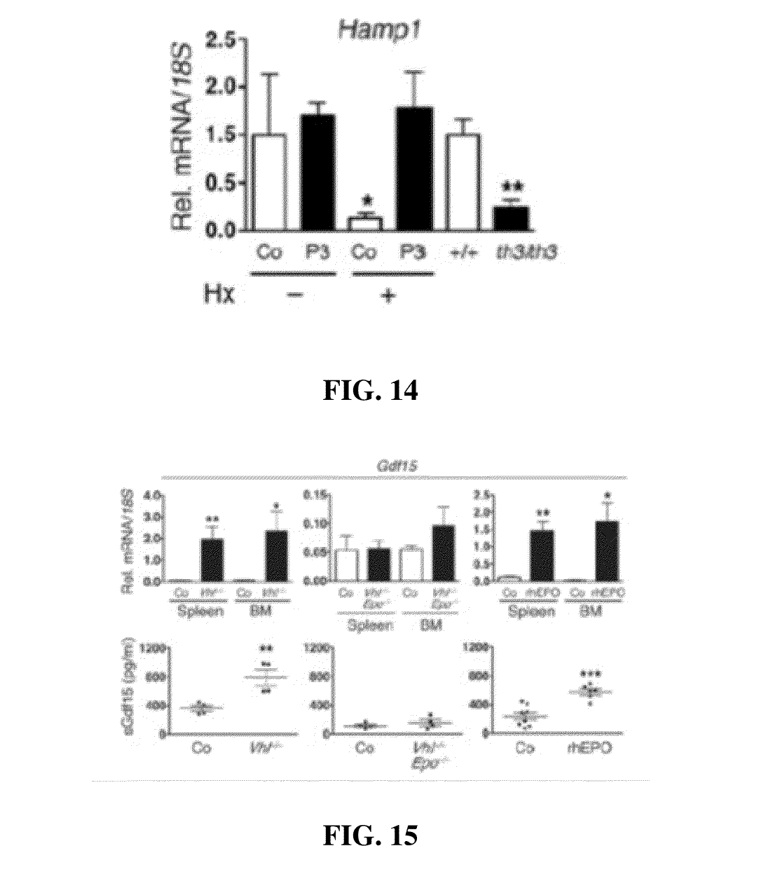

In order to gain additional insight into the role of Hif in the suppression of hepcidin under hypoxic conditions and its relation to erythropoietic activity, the inventors of the present disclosure compared mice with hypoproliferative anemia (Hif-2α/Pax3-cre (P3) mutants) that were exposed to chronic hypoxia to mice with hyperproliferative anemia (th3/th3 thalassemia mutants). Anemia in th3/th3 mutants is due to the elimination of both β-hemoglobin chains. Th3/th3 mice stabilize Hif-α in liver and kidney, and are characterized by high serum Epo levels, high erythropoietic activity, iron overload and substantial suppression of liver hepcidin (35). In contrast to th3/th3 mice, P3 mutants lack the ability to induce renal Epo in response to acute and chronic hypoxic stimuli. P3 mutant mice develop severe hypoproliferative anemia at baseline, are characterized by Hif-α stabilization in kidney and liver and display a blunted erythropoietic response when exposed to chronic hypoxia (10% O₂) for 10 days (30). It was found that hepcidin was not suppressed in P3 mutant livers compared to Cre⁻ control littermates under conditions of chronic hypoxia (FIG. 14), which is in contrast to th3/th3 mutants examined under baseline conditions. These findings in mice with two different forms of severe chronic anemia are in support of the notion that erythropoietic activity regulates hepcidin suppression under conditions of chronic hypoxia and/or Hif activation.

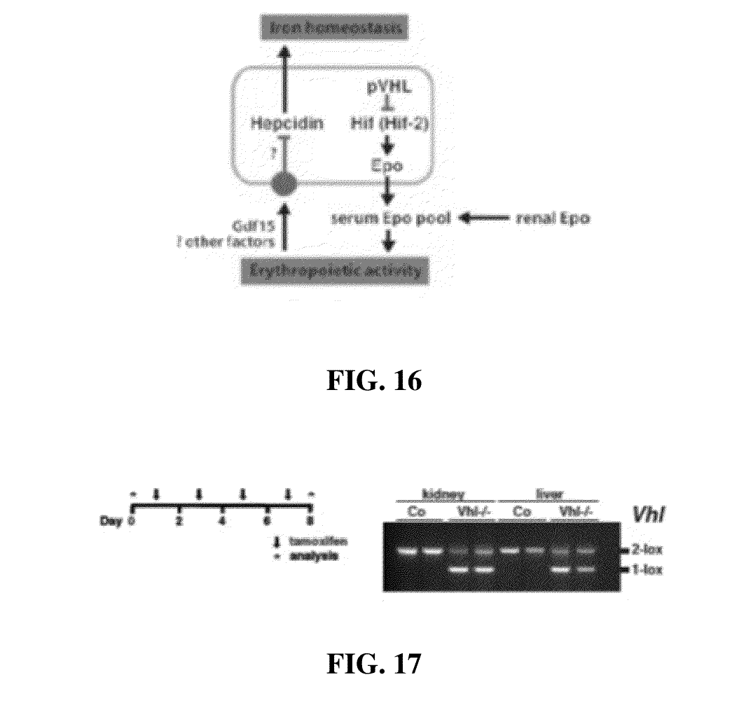

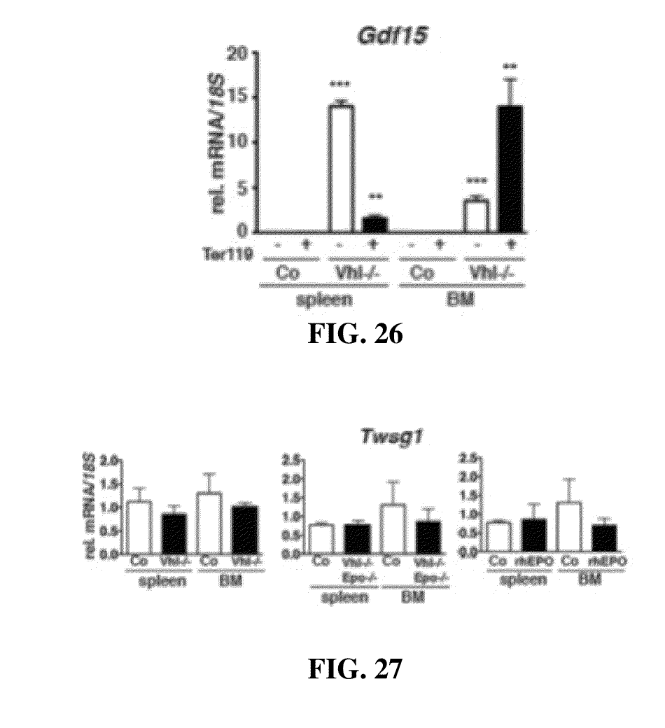

Since GDF15 has been proposed to be involved in hepcidin suppression at least under conditions of ineffective erythropoiesis, such as in patients with thalassemia syndromes (28), serum Gdf15 levels in Vhl^{-/-}, Vhl/Epo^{-/-} mice and in WT mice injected with recombinant human EPO were examined. The inventors of the present disclosure found that serum Gdf15 levels were increased in Vhl^{-/-} mice (789±108.5 pg/ml vs. 359.1±40.16 pg/ml) but not in Vhl/Epo double mutants compared to controls. A similar degree of Gdf15 increase was found when WT mice were treated with 3 daily injections of human recombinant EPO at a dose of 200 IU each (FIG. 15-16). EPO treatment was associated with hepcidin suppression (data not shown). Elevated serum Gdf15 levels correlated with increased Gdf15 mRNA expression in total cell isolates from spleen and bone marrow and in Ter119-positive cells purified by immunomagnetic separation (FIG. 15-16 and FIG. 26). In contrast to Gdf15, mRNA levels of twisted gastrulation homolog 1 (Twsg1), an erythrokine that has been shown to regulate hepcidin in vitro (36), did not change in Vhl mutants compared to littermate controls (FIG. 27). Taken together the data indicate that Gdf15 may participate in the suppression of hepcidin in Vhl^{-/-} mice.

To dissect the role of Hif in the regulation of hepcidin in vivo, the inventors of the present disclosure have generated a novel mouse model that permits dissociation of Epo synthesis from Hif activation. From using this model genetic evidence that hepcidin suppression requires Epo-induced erythropoiesis and is not directly regulated by either Hif-1 or Hif-2 is provided. Furthermore, it has been shown that the ability of the bone marrow to respond to elevated serum Epo levels with increased rbc production determines whether hepcidin is suppressed under conditions of Hif activation in the liver.

The importance of pO₂ in the regulation of hepcidin has been well established in cell culture models, in animal experiments and in humans, who were exposed to hypobaric hypoxia (7, 37). Its hypoxic regulation involves the VHL/HIF/PHD oxygen-sensing pathway, as shown in mouse models (8) and in patients with Chuvash polycythemia, a form of familial secondary erythrocytosis that associates with low serum hepcidin levels (5, 6). Chuvash patients are homozygous for specific non-tumor causing germ line mutations in the VHL tumor suppressor. These mutations impair the ability to efficiently degrade Hif-α under normoxia (5, 38). In line with laboratory findings in Chuvash patients is the Hif-dependent decrease of hepcidin in Vhl^{-/-} livers. Although Hif acts as an O₂-sensitive transcription factor, a direct transcriptional role for Hif was not evident, which is consistent with recently reported findings in hepatoma cell lines (9). While Hif-1 binding to the hepcidin promoter has been reported (8), stabilization of Hif-1α alone in Phd2^{-/-} hepatocytes did not result in a transcriptional repression of hepcidin. In this model, Hif-1 activation occurs without the induction of Epo synthesis, which is seen when Hif-2α is stabilized (32). Furthermore, the notion that hepcidin is not directly regulated by Hif-1 is consistent with genome-wide chromatin immunoprecipitation (ChIP) analysis in breast cancer cells, which indicates that HIF transcription factors are very unlikely to act as direct transcriptional repressors (39).

Although in cell lines, Hif has been reported to induce matriptase-2 (TMPRSS6) and furin, two proteases that modulate hepcidin expression by blunting BMP6/HJV signaling (16, 19), hepatic Hif activation without the concomitant increase in Epo transcription does not suppress hepcidin, which would argue against a regulatory role of furin and matriptase 2 in the inventors' model. This notion is furthermore supported by a lack of increase in Tmprss6 and furin mRNA levels in Vhl^{-/-} mice (FIG. 29). The ability to synthesize Epo was an absolute requirement for hepcidin suppression despite constitutive Hif-1 and Hif-2 activation in the liver. The data also argue against a direct role for Epo in the regulation of hepcidin and suggest that hepcidin suppression in Vhl^{-/-} livers is independent of hepatic EpoR activation. In the inventors' model of global Vhl deficiency, Epo synthesis is strongly enhanced in liver and kidney, and paracrine or autocrine activation of hepatocyte EpoR is unlikely to be involved in hepcidin regulation in vivo. During preparation of this manuscript, Mastrogiannaki and colleagues reported that treatment with anti-Epo blocking serum raised hepcidin mRNA levels in hepatocyte-specific Vhl/Hif-1α knock out mice (Albumin-cre model) to levels similar to those found in vehicle-injected control mice (40). This observation together with the inventors' findings are in contrast to in vitro data from human hepatoma cells and primary hepatocytes, where Epo has been shown to regulate hepcidin in a dose-dependent manner through activation of its receptor (21). While this discrepancy could be a reflection of differences between experimental approaches, i.e. cell culture studies versus whole animal models, the data are consistent with findings in severely anemic mice (anemia was induced by phlebotomy), which are characterized by low hepcidin expression (22). In their report, Pak and colleagues investigated whether anemia itself, elevated serum Epo or erythropoietic activity was required for hepcidin suppression. Treatment of anemic mice with Cp, doxorubicin or a non-cytotoxic Epo-blocking Ab inhibited erythropoiesis and raised hepcidin levels above normemic control levels (22), suggesting that hepcidin is not directly regulated by either Epo or tissue hypoxia under anemic conditions, but rather by a signal that is associated with increased erythropoietic activity. In keeping with the findings by Pak and colleagues in Cp-, doxorubicin- and anti-EPO Ab-treated mice, hepcidin levels were also significantly increased in Vhl/Hif-1/Hif-2^{-/-}, Vhl/Epo^{-/-} and Cp-treated control and Vhl^{-/-} mice, which are characterized by diminished or inhibited erythropoietic activity.

Serum iron has been shown to regulate hepcidin synthesis. Acute depletion of iron results in hepcidin suppression involving matriptase-2 (41), whereas iron loading increases hepcidin via TFR2-, HJV-, BMP6- and HFE-mediated signals (42). In the context of iron-deficiency anemia Hif-1α and Hif-2α are stabilized in Epo-producing tissues, primarily in kidney, but also in the liver depending on the severity of anemic hypoxia. This results in an increase of serum Epo levels and the suppression of hepcidin (1). Hif-2 is the main regulator of both renal and hepatic Epo synthesis under hypoxic conditions (30, 32) and does not appear to be involved in the regulation of hepcidin in hepatocytes (40). While changes in serum iron levels were not observed in mice with global Vhl deficiency at the time point(s) when analyses were performed, it was found that hepatic H-ferritin levels were reduced, which is suggestive of a decrease in intracellular free iron. The ferritin 5′-UTR contains an iron response element (IRE) that mediates translational inhibition in the presence of low intracellular iron. It is of interest to point out that H-ferritin reduction was dependent on the ability to synthesize Epo, but not on Vhl status nor the presence of stabilized Hif-α (H-ferritin was not reduced in Vhl/Epo^{-/-} livers). However, in certain VHL-deficient renal cancer cell lines H-ferritin and the labile iron pool were decreased (43). It is likely that the changes in H-ferritin levels seen in Vhl^{-/-} livers are a consequence of enhanced erythropoietic activity and iron utilization. Nevertheless, it cannot be excluded that altered intracellular iron levels have contributed to the regulation of hepcidin in the inventors' model. While liver tissue has not been examined, serum iron and ferritin levels are decreased in Chuvash patients and in individuals sojourning at high altitude for 10-12 days (5400 m) (6, 37). However, time course analysis showed that the decrease of serum hepcidin in subjects ascending to high altitude was rapid and preceded changes in serum ferritin and transferrin saturation. This observation suggests that iron-independent systemic signals must play a major role in the physiologic regulation of hepcidin under hypoxic conditions (44). This notion is supported by clinical observations in patients with β-thalassemia, who have low serum hepcidin levels in the presence of iron overload (45).

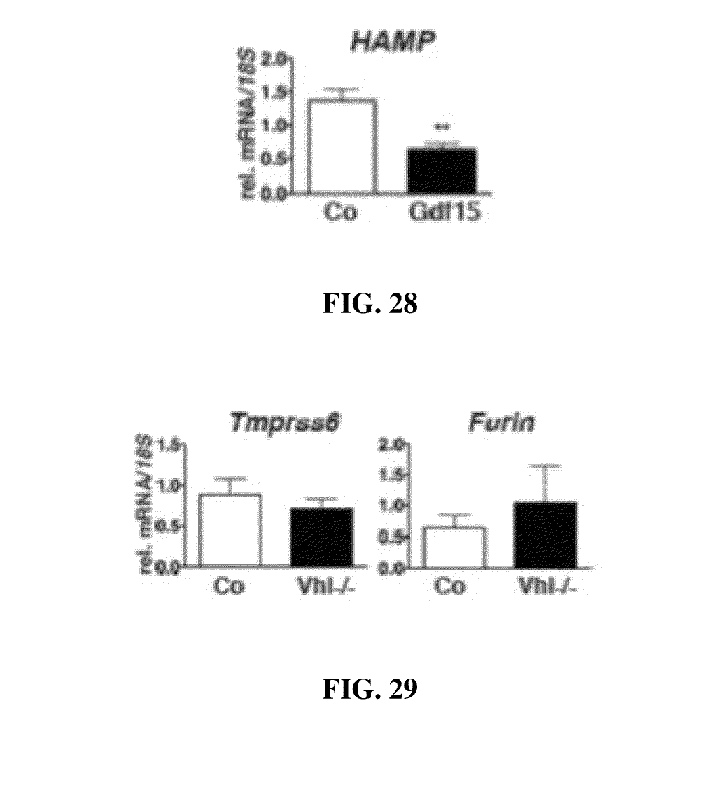

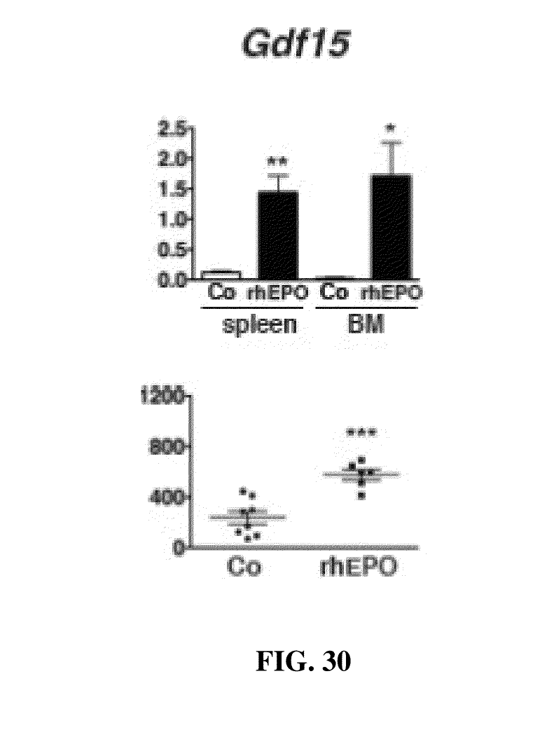

Recently GDF-15 and TWSG1 have been proposed to be erythroblast-derived factors, although not erythroblast-specific, that mediate hepcidin suppression under conditions of increased erythropoietic activity (24, 36). In particular, high levels of serum GDF15 associate with ineffective erythropoiesis, and may reflect a certain type of bone marrow stress or erythroblast apoptosis (28). The role of GDF15 in hepcidin regulation under physiologic conditions and in other pathologic settings, however, is unclear and has been debated. Whereas Twsgl mRNA expression levels did not change in bone marrow and spleen from Vhl^{-/-} mice, Gdf15 mRNA levels were elevated and were associated with increased serum concentrations of Gdf15. Although Gdf15 serum levels in Vhl^{-/-} mice were much lower (increased by approximately 2-fold over control) than those reported in β-thalassemia patients (mean of 66,000 pg/ml, (24)), the data from Hep3B cells exposed to smaller doses of recombinant Gdf15 support the hypothesis that Gdf15 may have contributed to hepcidin suppression in Vhl−/− mice. The present inventors found that recombinant murine Gdf15 suppressed hepcidin in Hep3B cells at a concentration of 750 pg/ml (FIG. 28). This is in contrast to previous reports where higher doses of GDF15 were needed to achieve hepcidin suppression in human HuH-7 hepatoma cells and in primary hepatocytes, while low dose GDF15 treatment increased hepcidin in these cells (24). The molecular basis of these differences in GDF15 dose responses is not clear and warrants further investigations. While the present inventors cannot completely exclude that Hif activation in hepatocytes modulates their response to Gdf15, elevation of serum Gdf15 in Vhl^{-/-} mice is not likely to result from Hif activation, as a similar degree of increase was found when wild type mice were treated with human recombinant EPO, which was also associated with hepcidin suppression. Studies in humans have not yet demonstrated a significant association between suppression of hepcidin levels and serum GDF15 levels following EPO administration (46), which may relate to the EPO doses used, study size, complexity of regulation and species-dependent differences in hepcidin regulation. In the context of iron-deficiency anemia, Tanno and colleagues reported that GDF15 serum levels were not elevated (47), while in a report by Lakhal and colleagues, patients with low serum iron had elevated GDF15 levels compared to iron-replete controls (mean of 1048 pg/ml vs. 542 pg/ml) (23). Similarly, increased serum GDF15 levels were found following DFO treatment, suggesting iron-dependent regulation (23). Temporary increases in serum GDF15 levels were also observed following ascent to high altitude, which associated with increases in serum EPO (44).

In summary, genetic means were used to dissect the role of Hif and Epo in the regulation of hepcidin and have shown that Hif-associated suppression of hepcidin occurs indirectly through Epo-induced erythropoiesis and may involve Gdf15 (FIG. 16). These data have implications for targeted therapies that aim at exploiting the VHL/HIF/PHD axis for the treatment of anemia and disorders of iron homeostasis.

Methods

Generation of mice and genotyping. The generation and genotyping of Vhl, Epo, Hif1a and Hif2a (Epos1) conditional alleles as well as Albumin-cre and Ubc-cre/ERT2 transgenes has been described elsewhere (29, 32, 34). Inducible Cre-mediated global inactivation of pVHL, Hif-1α, Hif-2α and/or Epo was achieved by generating mice that were homozygous for the Vhl, Hif1α, Hif2α and/or Epo conditional alleles and expressed a tamoxifen-inducible Cre-recombinase under control of the ubiquitin c promoter, Ubc-cre/ERT2 (29). The following genotypes were generated: (a) Vhl^{2lox/2lox}; Ubc-cre/ERT2, (b) Vhl^{2lox/2lox}; Hif1α^{2lox/2lox}; Hif2α^{2lox/2lox}; Ubc-cre/ERT2 and (c) Vhl^{2lox/2lox}; Epo^{2lox/2lox}; Ubc-cre/ERT2 referred to as Vhl^{-/-}, Vhl/Hif-1/Hif-2^{-/-} or Vhl/Epo^{-/-} after completion of tamoxifen treatment. For the temporary activation of the Ubc-cre/ERT2 transgenic system, mice received 4 i.p. injections of tamoxifen (Sigma-Aldrich) administered every other day at a concentration of 10 mg/ml (˜1.5 mg/mouse). Tamoxifen was dissolved in a mixture of 10 vol % ethanol and 90 vol % sunflower oil. Mice were phenotyped on day 8 after the first tamoxifen injection (outline of experimental protocol is shown in FIG. 17-21). Hepatocyte-specific inactivation of Phd2 (EglN1) was achieved by generating mice that expressed the Albumin-cre transgene and that were homozygous for the Phd2 conditional allele (EglN1^{2lox/2lox}; Albumin-cre), referred to as Albumin-Phd2 mutants. Cre-negative (Cre⁻) littermates from the same breeding pair were used as controls in all experiments. The generation and characterization of Hif2α/Pax3-cre mutant mice, referred to as P3 mutants, and thalassemic mice (th3/th3 mutants) has been described elsewhere (30, 35). Stefano Rivella, Weill Medical College of Cornell University, New York, N.Y. provided liver mRNAs from th3/th3 mutants and littermate controls.

All procedures involving mice were performed in accordance with NIH guidelines for the use and care of live animals and were reviewed and approved by the Institutional Animal Care and Use Committee (IACUC) of Vanderbilt University, Nashville, Tenn.

Phenotypic analysis of mutant mice: Hematocrits were determined by microcapillary tube centrifugation or with a Hemavet 950 analyzer (Drew Scientific). Serum Epo levels were determined by ELISA (R&D Systems); serum and liver iron concentrations were measured using the Iron Assay Kit from BioVision; serum Gdf15 concentrations were measured by ELISA (MyBioSource, LLC). Reticulocyte counts were determined by FACS analysis of whole blood stained with thiazole orange following the manufacturer's instructions (Sigma-Aldrich). For FACS analysis of bone marrow and spleen-derived erythroid precursor cells, 1×10⁶ bone marrow or spleen cells were incubated with PE-conjugated anti-transferrin receptor protein 1 (CD71) or FITC-conjugated anti-Ter119 monoclonal antibodies (BD Pharmingen) as previously described (50). For the analysis of Gdf15 mRNA levels Ter119-positive cells were isolated from bone marrow and spleen by immunomagnetic separation (Ter119-MicroBeads, Miltenyi Biotech). Recombinant human EPO (Amgen) was dissolved in 0.1 ml water and injected i.p. at a dose of 200 IU every day for 3 days prior to mouse phenotyping. For the pharmacologic inhibition of erythropoiesis, mice received a single i.p. injection of carboplatin (Sigma-Aldrich), 2.5 mg dissolved in 0.25 ml water one day before the first tamoxifen injection. Control animals received 0.25 ml of normal saline. For studies under conditions of chronic hypoxia, mice were exposed to 10% O₂ for 10 days in an animal hypoxia chamber (Biospherix Ltd) and analyzed immediately after hypoxia treatment.

DNA, RNA and protein analysis: For genotyping tail DNA was isolated according to Laird et al. (48). Other tissue DNA was isolated with DNeasy Blood & Tissue Kit according to the manufacturer's instructions (Qiagen). RNA was isolated using RNeasy Mini Kit according to the manufacturer's protocol (Qiagen). For real-time PCR analysis, 1 μl of cDNA was subjected to PCR amplification on an ABI 7300 platform using either SYBR Green PCR Master Mix or Taqman Universal PCR Master Mix (Applied Biosystems). Relative mRNA expression levels were quantified with the relative standard curve method according to the manufacturer's instructions (Applied Biosystems). 18S ribosomal RNA was used for normalization (49). Primer sequences for the analysis of Vhl, Vegf, Dmt1, Tfrc and Hamp1 have been published elsewhere (30, 32, 35). The following primer sequences were used for mRNA detection: Epo (forward, 5′-TGGTCTACGTAG CCTCACTTCACT-3′; reverse, 5′-TGGAGGCGACATCAATTC CT-3′); Gdf15 (forward, 5′-CAGAGCCGAGAGGACTCGAA-3′; reverse, 5′-CCGGTT GACGCGGAGTAG-3′); Twsg1 (forward, 5′-AGCATGCACTCCTTACAGCA-3′; reverse, 5′ -ACAAAGCACTCTGTGCCAGC-3′); Tmprss6 (forward, 5′-ACAGGGTGG CGATGTACGA-3′; reverse, 5′-GCACCCATAGACCGAGGTGAT-3′); furin (forward, 5′ -GTGCCTGCTCAGTGCCAG-3′; reverse, 5′-CGCTCGTCCGGAAAAGTT-3′); human hepcidin (forward, 5′-CAGCTGGATGCCCATGTTC-3′; reverse, 5′ -AGCCGCAGCAGAAAATGC-3′). Nuclear protein extracts for Western blot analysis were prepared and Hif-1α and Hif-2α were detected as previously described (32); H-ferritin and β-actin protein levels were analyzed with antibodies from Alpha Diagnostic International and Sigma-Aldrich.

Cell culture: Hep3B cells were cultured in DMEM supplemented with 10% FBS, recombinant Gdf15 (MyBioSource, LLC) was added to the culture medium to achieve a final concentration of 750 pg/ml.

Statistical analysis: Data reported represent mean values±S.E.M. Statistical analyses were performed with Prism 5.0b software (GraphPad Software) using the unpaired Student's t-test. For consistency, all P values reported were derived from unpaired 1-tailed Student's t-test analysis. P-values of <0.05 were considered statistically significant.

Throughout this document, various references are mentioned. All such references are incorporated herein by reference, including the references set forth in the following list:

### REFERENCES

1. Haase, V. H. Hypoxic regulation of erythropoiesis and iron metabolism. Am J Physiol Renal Physiol. 2010; 299(1):F1-13.
2. Andrews, N. C. Forging a field: the golden age of iron biology. Blood. 2008; 112(2):219-230.
3. Jelkmann, W. Erythropoietin after a century of research: younger than ever. Eur J Haematol. 2007; 78(3):183-205.
4. Kaelin, W. G., and Ratcliffe, P. J. Oxygen sensing by metazoans: the central role of the HIF hydroxylase pathway. Mol Cell. 2008; 30(4):393-402.
5. Ang, S. O., Chen, H., Hirota, K., et al. Disruption of oxygen homeostasis underlies congenital Chuvash polycythemia. Nature Genetics. 2002; 32(4):614-621.
6. Gordeuk, V. R., Miasnikova, G. Y., Sergueeva, A. I., et al. Chuvash polycythemia VHLR200W mutation is associated with down-regulation of hepcidin expression. Blood. 2011; 118(19):5278-5282.
7. Nicolas, G., Chauvet, C., Viatte, L., et al. The gene encoding the iron regulatory peptide hepcidin is regulated by anemia, hypoxia, and inflammation. J Clin Invest. 2002; 110(7):1037-1044.
8. Peyssonnaux, C., Zinkernagel, A. S., Schuepbach, R. A., et al. Regulation of iron homeostasis by the hypoxia-inducible transcription factors (HIFs). J Clin Invest. 2007; 117(7):1926-1932.
9. Volke, M., Gale, D. P., Maegdefrau, U., et al. Evidence for a lack of a direct transcriptional suppression of the iron regulatory peptide hepcidin by hypoxia-inducible factors. PLoS ONE. 2009; 4(11):e7875.
10. Braliou, G. G., Verga Falzacappa, M. V., Chachami, G., Casanovas, G., Muckenthaler, M. U., and Simos, G. 2-Oxoglutarate-dependent oxygenases control hepcidin gene expression. J Hepatol. 2008; 48(5):801-810.
11. Gao, J., Chen, J., Kramer, M., Tsukamoto, H., Zhang, A. S., and Enns, C. A. Interaction of the hereditary hemochromatosis protein HFE with transferrin receptor 2 is required for transferrin-induced hepcidin expression. Cell Metab. 2009; 9(3):217-227.
12. Nemeth, E., and Ganz, T. The role of hepcidin in iron metabolism. Acta Haematol. 2009; 122(2-3):78-86.
13. Babitt, J. L., Huang, F. W., Wrighting, D. M., et al. Bone morphogenetic protein signaling by hemojuvelin regulates hepcidin expression. Nat Genet. 2006; 38(5):531-539.
14. Andriopoulos, B., Jr., Corradini, E., Xia, Y., et al. BMP6 is a key endogenous regulator of hepcidin expression and iron metabolism. Nat Genet. 2009; 41(4):482-487.
15. Meynard, D., Kautz, L., Darnaud, V., Canonne-Hergaux, F., Coppin, H., and Roth, M. P. Lack of the bone morphogenetic protein BMP6 induces massive iron overload. Nat Genet. 2009; 41(4):478-481.
16. Silvestri, L., Pagani, A., and Camaschella, C. Furin-mediated release of soluble hemojuvelin: a new link between hypoxia and iron homeostasis. Blood. 2008; 111(2):924-931.
17. McMahon, S., Grondin, F., McDonald, P. P., Richard, D. E., and Dubois, C. M. Hypoxia-enhanced expression of the proprotein convertase furin is mediated by hypoxia-inducible factor-1: impact on the bioactivation of proproteins. J Biol Chem. 2005; 280(8):6561-6569.
18. Du, X., She, E., Gelbart, T., et al. The serine protease TMPRSS6 is required to sense iron deficiency. Science. 2008; 320(5879):1088-1092.
19. Lakhal, S., Schodel, J., Townsend, A. R., Pugh, C. W., Ratcliffe, P. J., and Mole, D. R. Regulation of type II transmembrane serine proteinase TMPRSS6 by hypoxia-inducible factors: new link between hypoxia signaling and iron homeostasis. J Biol Chem. 2010; 286(6):4090-4097.
20. Silvestri, L., Pagani, A., Nai, A., De Domenico, I., Kaplan, J., and Camaschella, C. The serine protease matriptase-2 (TMPRSS6) inhibits hepcidin activation by cleaving membrane hemojuvelin. Cell Metab. 2008; 8(6):502-511.
21. Pinto, J. P., Ribeiro, S., Pontes, H., et al. Erythropoietin mediates hepcidin expression in hepatocytes through EPOR signaling and regulation of C/EBPalpha. Blood. 2008; 111(12):5727-5733.
22. Pak, M., Lopez, M. A., Gabayan, V., Ganz, T., and Rivera, S. Suppression of hepcidin during anemia requires erythropoietic activity. Blood. 2006; 108(12):3730-3735.
23. Lakhal, S., Talbot, N. P., Crosby, A., et al. Regulation of growth differentiation factor 15 expression by intracellular iron. Blood. 2009; 113(7):1555-1563.
24. Tanno, T., Bhanu, N. V., Oneal, P. A., et al. High levels of GDF15 in thalassemia suppress expression of the iron regulatory protein hepcidin. Nat Med. 2007; 13(9):1096-1101.
25. Tamary, H., Shalev, H., Perez-Avraham, G., et al. Elevated growth differentiation factor 15 expression in patients with congenital dyserythropoietic anemia type I. Blood. 2008; 112(13):5241-5244.
26. Ramirez, J. M., Schaad, O., Durual, S., et al. Growth differentiation factor 15 production is necessary for normal erythroid differentiation and is increased in refractory anaemia with ring- sideroblasts. Br J Haematol. 2009; 144(2):251-262.
27. Finkenstedt, A., Bianchi, P., Theurl, I., et al. Regulation of iron metabolism through GDF15 and hepcidin in pyruvate kinase deficiency. Br J Haematol. 2009; 144(5):789-793.
28. Tanno, T., Noel, P., and Miller, J. L. Growth differentiation factor 15 in erythroid health and disease. Curr Opin Hematol. 2010; 17(3):184-190.
29. Ruzankina, Y., Pinzon-Guzman, C., Asare, A., et al. Deletion of the developmentally essential gene ATR in adult mice leads to age-related phenotypes and stem cell loss. Cell Stem Cell. 2007; 1(1):113-126.
30. Kapitsinou, P. P., Liu, Q., Unger, T. L., et al. Hepatic HIF-2 regulates erythropoietic responses to hypoxia in renal anemia. Blood. 2010; 116(16):3039-3048.
31. Rankin, E. B., Rha, J., Selak, M. A., et al. Hypoxia-inducible factor 2 regulates hepatic lipid metabolism. Mol Cell Biol. 2009; 29(16):4527-4538.
32. Rankin, E. B., Biju, M. P., Liu, Q., et al. Hypoxia-inducible factor-2 (HIF-2) regulates hepatic erythropoietin in vivo. J Clin Invest. 2007; 117(4):1068-1077.
33. Minamishima, Y. A., and Kaelin, W. G., Jr. Reactivation of hepatic EPO synthesis in mice after PHD loss. Science. 2010; 329(5990):407.
34. Zeigler, B. M., Vajdos, J., Qin, W., Loverro, L., and Niss, K. A mouse model for an erythropoietin-deficiency anemia. Dis Model Mech. 2011; 3(11-12):763-772.
35. Gardenghi, S., Marongiu, M. F., Ramos, P., et al. Ineffective erythropoiesis in beta-thalassemia is characterized by increased iron absorption mediated by down-regulation of hepcidin and up-regulation of ferroportin. Blood. 2007; 109(11):5027-5035.
36. Tanno, T., Porayette, P., Sripichai, O., et al. Identification of TWSG1 as a second novel erythroid regulator of hepcidin expression in murine and human cells. Blood. 2009; 114(1):181-186.
37. Piperno, A., Galimberti, S., Mariani, R., et al. Modulation of hepcidin production during hypoxia-induced erythropoiesis in humans in vivo: data from the HIGHCARE project. Blood. 2011; 117(10):2953-2959.
38. Hickey, M. M., Lam, J. C., Bezman, N. A., Rathmell, W. K., and Simon, M. C. von Hippel-Lindau mutation in mice recapitulates Chuvash polycythemia via hypoxia-inducible factor-2alpha signaling and splenic erythropoiesis. J Clin Invest. 2007; 12(117):3879-3889.
39. Schodel, J., Oikonomopoulos, S., Ragoussis, J., Pugh, C. W., Ratcliffe, P. J., and Mole, D. R. High-resolution genome-wide mapping of HIF-binding sites by ChIP-seq. Blood. 2011; 117(23):e207-217.
40. Mastrogiannaki, M., Matak, P., Mathieu, J. R., et al. Hepatic HIF-2 down-regulates hepcidin expression in mice through epo-mediated increase in erythropoiesis. Haematologica. 2012; 97(6):824-834.
41. Zhang, A. S., Anderson, S. A., Wang, J., et al. Suppression of hepatic hepcidin expression in response to acute iron deprivation is associated with an increase of matriptase-2 protein. Blood. 117(5):1687-1699.
42. Ramos, E., Kautz, L., Rodriguez, R., et al. Evidence for distinct pathways of hepcidin regulation by acute and chronic iron loading in mice. Hepatology. 2011; 53(4):1333-1341.
43. Alberghini, A., Recalcati, S., Tacchini, L., Santambrogio, P., Campanella, A., and Cairo, G. Loss of the von Hippel Lindau tumor suppressor disrupts iron homeostasis in renal carcinoma cells. J Biol Chem. 2005; 280(34):30120-30128.
44. Talbot, N. P., Lakhal, S., Smith, T. G., et al. Regulation of hepcidin expression at high altitude. Blood. 2012; 119(3):857-860.
45. Ginzburg, Y., and Rivella, S. beta-thalassemia: a model for elucidating the dynamic regulation of ineffective erythropoiesis and iron metabolism. Blood. 2011; 118(16):4321-4330.
46. Ashby, D. R., Gale, D. P., Busbridge, M., et al. Erythropoietin administration in humans causes a marked and prolonged reduction in circulating hepcidin. Haematologica. 2009; 95(3):505-508.
47. Tanno, T., Rabel, A., Lee, Y. T., Yau, Y. Y., Leitman, S. F., and Miller, J. L. Expression of growth differentiation factor 15 is not elevated in individuals with iron deficiency secondary to volunteer blood donation. Transfusion. 2010; 50(7):1532-1535.
48. Laird, P. W., Zijderveld, A., Linders, K., Rudnicki, M.A., Jaenisch, R., and Berns, A. Simplified mammalian DNA isolation procedure. Nucleic Acids Res. 1991; 19(15):4293.
49. Rankin, E. B., Higgins, D. F., Walisser, J. A., Johnson, R. S., Bradfield, C. A., and Haase, V. H. Inactivation of the arylhydrocarbon receptor nuclear translocator (Arnt) suppresses von Hippel-Lindau disease-associated vascular tumors in mice. Mol Cell Biol. 2005; 25(8):3163-3172.
50. Liu, Y., Pop, R., Sadegh, C., Brugnara, C., Haase, V. H., and Socolovsky, M. Suppression of Fas-FasL coexpression by erythropoietin mediates erythroblast expansion during the erythropoietic stress response in vivo. Blood. 2006; 108(1):123-133.
51. Liu, Q., Davidoff, O., Niss, K., Haase, V. H., Hypoxia-inducible factor regulates hepcidin via erythropoietin-induced erythropoiesis. J. Clin. Invest. 2012; 122(12): 4635-44.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating a disorder of iron homeostasis in a subject, comprising:
providing a biological sample from the subject;
determining an amount of growth differentiation factor 15 (GDF15) in the sample;
comparing the amount of the GDF15 to a reference amount of GDF15; and
administering to the subject an effective amount of at least one of iron and erythropoietin (EPO) if there is a measurable difference in the amount of GDF15 in the sample as compared to the reference amount of GDF15.

2. The method of claim 1, wherein the effective amount of at least one of iron and EPO is administered to the subject on an hourly basis.

3. The method of claim 1, wherein the effective amount of at least one of iron and EPO is administered to the subject on a daily basis.

4. The method of claim 1, wherein the effective amount of at least one of iron and EPO is administered to the subject on a weekly basis.

5. The method of claim 1, wherein the disorder of iron homeostasis is anemia.

6. The method of claim 1, further comprising monitoring a bone marrow and/or an erythroid response in the subject.

7. The method of claim 1, wherein the subject is receiving dialysis.

8. The method of claim 1, wherein the subject is resistant to treatment with recombinant EPO and/or is not responding to treatment with recombinant EPO.

9. The method of claim 1, wherein the subject is receiving treatment with recombinant erythropoietin, a hypoxia-inducible factor (HIF)-stabilizing composition, and/or a composition that stimulates endogenous EPO synthesis.

10. The method of claim 1, wherein the sample is a blood sample or a serum sample.

11. The method of claim 1, further comprising determining a red blood cell count in a sample obtained from the subject.

12. The method of claim 1, wherein the subject is a human.

13. The method of claim 1, further comprising determining an amount of hepcidin in the sample.

14. The method of claim 13, further comprising comparing the amount of hepcidin in the sample to a reference amount of hepcidin.

15. The method of claim 13, further comprising determining a ratio of the amount of GDF15 in the sample to the amount of hepcidin in the sample.

16. A method for treating disorder of iron homeostasis in a subject, comprising:
providing a biological sample from the subject;
determining an amount of hepcidin in the sample;
comparing the amount of the hepcidin to a reference amount of hepcidin; and
administering to the subject an effective amount of at least one of iron and erythropoietin (EPO) if there is a measurable difference in the amount of hepcidin in the sample as compared to the reference amount of hepcidin.

17. The method of claim 16, wherein the effective amount of at least one of iron and EPO is administered to the subject on an hourly basis.

18. The method of claim 16, wherein the effective amount of at least one of iron and EPO is administered to the subject on a daily basis.

19. The method of claim 16, wherein the effective amount of at least one of iron and EPO is administered to the subject on a weekly basis.

20. The method of claim 16, wherein the disorder of iron homeostasis is anemia.
